(19) United States
(12) Patent Application Publication
(10) Pub. No.: US 2017/0354729 A1
(43) Pub. Date: Dec. 14, 2017

(54) VACCINE COMPOSITIONS CONTAINING MODIFIED ZIKA VIRUS ANTIGENS

(71) Applicant: NOVAVAX, INC., Gaithersburg, MD (US)

(72) Inventors: Ye LIU, Gaithersburg, MD (US); Jing-Hui TIAN, Gaithersburg, MD (US); Gale SMITH, Gaithersburg, MD (US); Michael J. MASSARE, Gaithersburg, MD (US); Sarathi BODDAPATI, Gaithersburg, MD (US)

(21) Appl. No.: 15/460,775

(22) Filed: Mar. 16, 2017

Related U.S. Application Data
(60) Provisional application No. 62/309,216, filed on Mar. 16, 2016.
(60) Provisional application No. 62/407,887, filed on Oct. 13, 2016.
(60) Provisional application No. 62/420,941, filed on Nov. 11, 2016.
(60) Provisional application No. 62/439,374, filed on Dec. 27, 2016.

(51) Int. Cl. A61K 39/12; C12N 7/00; C07K 14/005; A61K 39/00

(52) U.S. Cl. CPC A61K 39/12; C07K 14/005; C12N 7/00; A61K 2039/55577; C12N 2770/24122; C12N 2770/24131

(57) ABSTRACT

The present disclosure relates to vaccine compositions that comprise a Zika virus antigen and an adjuvant. The present disclosure also provides methods for inducing a protective immune response by administering the disclosed vaccine compositions in a subject in needs thereof. The present methods also comprise the binding of the Zika virus vaccine to Zika virus cellular receptor proteins.

### INCORPORATION BY REFERENCE

U.S. provisional application No. 62/309,216, filed on Mar. 16, 2016, 62/407,887, filed on Oct. 13, 2016, 62/420,941, filed on Nov. 11, 2016, and 62/439,374, filed Dec. 27, 2016, are each incorporated herein by reference in their entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: NOVV_070_03US_SeqList_ST25.txt, date recorded: Mar. 16, 2017; file size: 60 kilobytes).

### FIELD OF THE INVENTION

The present disclosure relates to vaccine compositions comprising a Zika virus antigen and, optionally, an adjuvant in an amount effective to enhance the immune response. The vaccine compositions are useful for inducing immune responses. The present disclosure also provides methods for inducing protective immune responses in subjects administered with the present vaccine compositions, as well as manufacturing the compositions.

### BACKGROUND

Infectious diseases remain a problem throughout the world. Zika virus has recently become a threat to public health and has rose to prominence as a potential cause of microcephaly, and other developmental defects resulting from infection during the early stages of pregnancy.

No zika vaccine is available and there is continuing interest in producing vaccines against viruses, such as zika, that present public health issues throughout the globe. In addition, there remains an ongoing need to produce effective vaccines with good stability.

### SUMMARY OF THE INVENTION

The present disclosure provides vaccine compositions comprising a Zika virus antigen. In some aspects, the vaccine compositions comprise an adjuvant. In some aspects, the Zika virus antigen is a secreted Zika envelope protein. The envelope protein is typically produced and administered as a dimer, which provides an enhanced immune response compared to a non-dimer formulations.

In some aspects, the adjuvant is selected from the group consisting of a mineral compound-based adjuvant, a bacterial adjuvant, an oil-based emulsion, an immunostimulatory complex (ISCOM), and a synthetic adjuvant. In a preferred aspect, the adjuvant is a Matrix-M adjuvant. In other aspects, the vaccine formulations further comprise a pharmaceutically acceptable carrier. In other aspects, the vaccine compositions comprise a Zika virus antigen and a Matrix-M adjuvant. Unless otherwise specified, the Matrix-M adjuvant referred to herein is Matrix-M1.

The present disclosure also provides methods of inducing a protective immune response in a subject, particularly a human. In some aspects, the methods comprise administering Zika virus vaccine compositions. In some aspects, the vaccine compositions administered to the subjects comprise a Zika virus antigen and an adjuvant. In a preferred aspect, the vaccine compositions administered to the subject comprise a Zika virus antigen and a Matrix-M adjuvant.

The immune response may comprise increased neutralizing antibody levels in the subjects. In aspects, the immune response comprises increased IgG levels in the subjects. As used herein, the subjects are humans. In some aspects, the subject is male; in other aspects; the subject is a human female; for example, a pregnant human female.

As used herein, the term “about” refers to plus or minus ten percent of the object that “about” modifies.

### BRIEF DESCRIPTION OF THE FIGURES

FIGS. 1A and 1B illustrate exemplary vaccine compositions disclosed herein.

FIG. 1 shows a cartoon of a ZIKV Envelope protein showing several domains (FIG. 1A) and a modified structure of a protein as expressed (FIG. 1B) illustrating a cleavage site and tag, each of which is optional as disclosed herein. Where present initially, part or all of a tag and/or part of all of a protease cleavage site may optionally be removed during processing. Figure key: BV: baculovirus; ZIKV: Zika virus; PrM: propeptide-Membrane; EnvD: N terminal 80% of E protein.

FIG. 2 illustrates a DNA sequence (SEQ ID NO:11) encoding a modified ZIKV envelope gene, where the expressed protein contains PrM.EnvD (67-69 DMA-NTT).His6.

FIG. 3A illustrates a Zika amino acid sequence disclosed herein for a modified ZIKV envelope protein expressed by baculovirus clone BV1993 PrM.EnvD (67-69 DMA-NTT).TEV.His6 (SEQ ID NO: 1). Key: Underline: PrM (SEQ ID NO:9); EnvD portion starts at IRC and ends at RSG; Star: TEV protease cleavage site (ENLYFQG; SEQ ID NO:2) and poly-His tag; TEV: Tobacco Etch Virus; His6: Poly-Histidine tag. The initiating “M” residue at position 1 is artificial. FIG. 3B illustrates an amino acid sequence (SEQ ID NO:3) of a modified ZIKV envelope gene encoded by BV 2002 PrM.EnvD (67-69 DMA-NTT).TEV.His6. Key: Underline: Pre-membrane (PrM); Bold: EnvD; and poly-His tag; His6: Poly-Histidine tag. The initiating “M” residue at position 1 is artificial. FIG. 3B lacks an introduced protease cleavage site.

FIG. 4 illustrates an amino acid sequence of a soluble, glycosylated Zika virus secreted envelope vaccine produced from the PrM.EnvD construct (SEQ ID NO:4). This amino acid sequence is the sequence of a mature peptide after PrM cleavage in Sf9 cells and TEV protease cleavage during purification to remove poly histidine and with the added N-linked glycosylation site (67-69 DMA to NTT). Key: ZIKV: Zika virus; TEV: Tobacco Etch Virus. The remaining portion of the TEV cleavage site is highlighted by the asterisk

FIG. 5A shows a Coomassie blue stained reduced SDS-PAGE, anti-ZIKV E western blot, and densitometry purity analysis for EnvD purified vaccine drug substance. Baculovirus (BV1944) expressing ZIKV PrM and ectodomain EnvD, (aa1-404) as precursor protein was used to infect 519 cells. ZIKV Env wild type glycosylation site N154 and the engineered glycosylation site N67 are labeled. After cellular protease cleavage between PrM and EnvD, mature EnvD was secreted into culture medium and purified.

FIG. 5B Dynamic light scattering (DLS) analysis of ZIKV EnvD. Purified ZIKV EnvD was analyzed using a Wyatt Dynapro Platereader DLS system equilibrated at 20° C. Light scattering was detected at 150° relative to the incident beam of monochromatic light (819.1 nm). The intensity-weighted particle distribution is shown, and is based on cumulant analysis of the experimental autocorrelation function (Inset: experimental data are blue; fit from cumulant analysis is brown and almost completely overlaps with the blue fit). The data shows the EnvD protein is dimeric.

FIG. 5C shows a sedimentation velocity analytical ultracentrifugation (SV AUC). SV AUC analysis of an exemplary purified ZIKV EnvD protein. SV AUC was performed on a Beckman Coulter ProteomeLab XL-I operated at 20° C. with a rotor speed of 45,000 rpm. Protein sedimentation was detected at a wavelength of 280 nm over the course of 6 h (Inset: experimental data are red; model shown in black). Data were analyzed using Ultrascan software. Results from modeling the data as a discrete distribution of sedimenting species are shown.

FIG. 5D illustrates the dynamic light scattering analysis on ZIKV EnvD dimer from a different batch than in FIG. 5B, illustrating reproducibility. The hydrodynamic radius was 4 nm and the estimated molecular weight of the ZIKV EnvD dimer protein was 86.3 kDa. Malvern Zetasizer Software V 7.11 was used for the data analysis. FIG. 5D illustrates the scanning densitometry of purified BV1944 EnvD protein. 93% of the ZIKV protein had a size of about 50 kDa.

FIG. 6A shows the ELISA titer results of a various ZIKV vaccines against the Zika virus infected cell lysate on Day 42 of the mouse study described in Example 1. ELISA plates were coated with Zika virus infected cell lysate and treated with one of the treatment groups (Group 1: HA1-sE (secreted Envelope) fusion protein vaccine, Group 2: HA1-sE fusion protein vaccine+Matrix-M™ adjuvant, Group 3: ZIKV sE secreted protein vaccine (BV1903), Group 4: ZIKV EnvD secreted protein vaccine (BV1903, which lacks the introduced glycosylation site)+Matrix-M™ adjuvant, Group 5: ZIKV iE (insoluble Envelope) refolded protein vaccine, Group 6: ZIKV iE refolded protein vaccine+Matrix-M™ adjuvant). The ELISA titers were based on four parameter fit analysis of antibody binding to the virus antigen in the Zika virus cellular lysate. The ZIKV EnvD secreted protein vaccine with a Matrix-M™ adjuvant had the highest titers, about 100-fold higher than the HA1-sE fusion protein vaccine and 500-fold higher than the ZIKV iE refolded protein vaccine. The ZIKV EnvD secreted protein vaccine was produced with BV1903. Key: HAL hemagglutinin; sE: secreted envelope; ZIKV: Zika virus; ELISA: enzyme-linked immunosorbent assay. FIG. 6B shows extended timepoint data for the mouse study using PrM.EnvD.His6 from BV1903. Day 42 data was presented in FIG. 6A as Group 3. Day 69 data for FIG. 6B shows maintained Anti-Zika IgG antibodies. GMT for each group is represented with the black bar. Error bars indicate 95% confidence intervals.

FIG. 7A shows the mircroneutralizing (MN50) antibody titers of a ZIKV vaccine against the Zika virus infected cell lysate on Day 42 of the mouse study described in Example 1. The cell lysate was treated with one of the treatment groups (Group 1: HA1-sE fusion protein vaccine, Group 2: HA1-sE fusion protein vaccine+Matrix-M™ adjuvant, Group 3: ZIKV sE secreted protein vaccine (BV1903), Group 4: ZIKV sE secreted protein vaccine (BV1903)+Matrix-M™ adjuvant, Group 5: ZIKV iE refolded protein vaccine, Group 6: ZIKV iE refolded protein vaccine+Matrix-M™ adjuvant). The ZIKV sE secreted protein vaccine with a Matrix-M™ adjuvant had the highest titers, about 50-fold higher than both the HA1-sE fusion protein vaccine and ZIKV iE refolded protein vaccine and about 20-fold above the reported protective level. The ZIKV sE secreted protein vaccine was produced with BV1903. Key: HAL hemagglutinin; sE: secreted envelope; ZIKV: Zika virus. FIG. 7B show extended timepoint data for the mouse study using PrM.EnvD.His6, the protein expressed from BV1903. For neutralizing antibodies, Day 42 data was presented in FIG. 7A. Day 69 data for FIG. 7B shows maintained neutralizing antibody production. Individual animal response is shown with each symbol. GMT for each group is represented with the black bar. Error bars indicate 95% confidence intervals.

FIG. 8 shows the binding of human Zika convalescent serum to the three forms of ZIKV envelope protein (HA1-sE fusion protein vaccine, ZIKV sE secreted protein vaccine, and ZIKV iE refolded protein vaccine). ZIKV sE (prM-EnvD, BV1903) bound with higher titer and avidity to human convalescent serum. Relative binding was predictive of the induction of functional immunity. Key: ZIKV: Zika virus; sE: secreted envelope protein; HA: hemagglutinin.

FIG. 9 shows the binding kinetics of the vaccine protein produced from Zika BV1903 and Zika BV1944, which has an introduced glycosylation site at amino acids 67-69, to human convalescent serum in ELISA assay.

FIG. 10 shows the binding of BV1944 ZIKV sE (FIG. 10A) and BV1858 ZIKV iE (FIG. 10B) to human Zika convalescent IgG and mouse antibody 4G2.

FIG. 11 shows the diagram of the binding kinetics of BV1944 ZIKV sE (FIG. 11A) and BV1858 ZIKV iE (FIG. 11B) to AXL and DC-SIGN. Failure to bind to AXL and DC-SIGN shows that BV1858 is improperly folded.

FIG. 12 shows binding of anti-EDE1 antibodies to ZIKV protein from BV1944. Binding curves were obtained by passing different concentration, as indicated, over biosensor chips on which the anti-EDE1 mAb C8 (left panel) or anti-EDE1 mAb C10 (right panel) were immobilized. Kinetic values were obtained by fitting the association and dissociation responses to a 1:1 binding model

FIGS. 13A-13D shows immune response characterization of an EnvD Zika vaccine composition disclosed herein. FIG. 13A shows a time-course of the ELISA titer responses at 20 and 46 days. plaque-reduction neutralization tests (“PRNT”) against Zika were performed to identify neutralizing antibodies. (See World Health Organization Department of Immunization Vaccines Biologicals. 2007. Guidelines for plaque-reduction neutralization testing of human antibodies to dengue viruses. World Health Organization, Geneva, Switzerland. WHO/IVB/07.07) FIG. 13B shows the results for Groups 1-3 plaque-reduction neutralization tests (“PRNT”) against Zika were performed to identify neutralizing antibodies. By day 20 a response was observed for both adjuvanted groups, Groups 2 and 3. At day 46 both Groups showed elevated neutralizing antibodies. Cross neutralization against two strains of Dengue (DENV-2 and DENV-4) were also demonstrated. FIG. 13C shows the ZIKV EnvD induces neutralization antibodies against Dengue-2. FIG. 13D shows the ZIKV EnvD induces neutralization antibodies against Dengue-4. For both DENV-2 and DENV-4 Matrix-M1 showed substantially greater production of neutralizing antibodies.

FIGS. 14A-14B shows immune response characterization of an EnvD Zika vaccine composition disclosed herein in a Rhesus macaque model of ZIKV infection. FIG. 14A shows ELISA data for each of the five groups. Neutralization data using the PRNT assay is shown in FIG. 14B. For the PRNT experiments, a neutralization titer of 20 is considered protective in monkey challenge studies with Zika virus. Groups 3 and 4 exceeded this neutralization titer by week 6

FIG. 15 shows Zika dimer stability over time in a formulation containing PS20 and EDTA. SPR data is shown at 4° C. and 25° C. with different concentrations of Matrix M adjuvant.

FIG. 16 shows Zika dimer stability over time in a formulation without PS20 and without EDTA. SPR data is shown at 4° C. and 25° C. with different concentrations of Matrix M adjuvant.

### DETAILED DESCRIPTION

Disclosed herein are vaccine compositions that comprise Zika virus antigen and, optionally, an adjuvant. The present disclosure also provides methods for inducing protective immune responses by administering the vaccine compositions as described herein. (Plevka et al., “Maturation of flaviviruses starts from one or more icosahedrally independent nucleation centres,” Int. J. Infect. Dis. 2016; 44: 11-15.)

ZIKV displays a similar structure to other known flaviviruses. For flavivirus, mature virus particles contain 180 copies of the E protein (also known as “Env”) and membrane (M) protein on the envelope and display an icosahedral arrangement in which 90 E dimers completely cover the viral surface. Upon entry into host cells via endocytosis, the acidic endosomal environment triggers an irreversible conformational change in the E protein and a transition from a dimer to trimer formation that leads to the membrane fusion event. In the ER, newly assembled virus progeny form immature virions and exhibit a spiky surface anchored with 60 trimeric protrusions of the E and precursor-membrane (prM) heterodimers. During virus maturation, a low pH environment in the trans-Golgi network (TGN) induces the reorganization of the E-prM heterodimers into E homodimers (Yu et al., “Structure of the immature dengue virus at low pH primes proteolytic maturation,” Science. 2008 Mar. 28; 319(5871):1834-7). This structural rearrangement exposes the cleavage site of prM for digestion by the host protease, furin. After prM is cleaved, the protein dissociates from the particles upon release into the pH neutral extracellular space. During the maturation process, not all of E-prM heterodimer can be cleaved by the furin, and then uncleaved E-prM heterodimer will revert back to a spiky immature trimeric structure (Yu et al. 2008).

Stable E proteins have not been described to date. In addition, while the full-length protein, expressed as a DNA vaccine, has been demonstrated to provide protective efficacy; other variants have been shown to fail to protect from infection and only the full-length Env protein has been selected for further study. Larocca et al., Nature. 2016 Aug. 25; 536(7617):474-8; Abbink et al., Science. 2016 Sep. 9; 353(6304):1129-32. It has been surprisingly discovered, however, that truncated versions of the Env protein can be prepared in stable form as dimers and used to prepare compositions that induce protective immune responses.

### Zika Virus Polypeptide Antigens

In one embodiment, Zika E polypeptide used herein may be derived from strain ZikaSPH2015 (See Genbank Accession number ALU33341.1 for the polyprotein sequence; SEQ ID NO: 5). Env proteins in other strains (including Genbank Accession number AIC06934.1) may also be used as sources of E proteins. Structurally, with reference to SEQ ID NO: 5, the polyprotein contains the propeptide “Pr” at amino acids 125 to 215, a membrane protein “M” at 216 to 290 (together referred to as “PrM”), and the full length Envelope protein (also referred to herein as “Env” or “E” protein) at 291-795. Proteins selected for vaccine compositions are truncated versions of the full length Env protein that do not exist in nature. Preferred portions of the E protein contain amino acids 291 to 694 of SEQ ID NO: 5. In certain embodiments, the E protein in the vaccine composition consists of amino acids 291 to 694 of SEQ ID NO: 5. This protein contains about 80% of the N-terminus of the Env ectodomain and lacks the stem and the TM domains and may be referred to herein as “E80,” “E80ΔStem” or “EnvD,” where, in particular contexts, EnvD may refer to dimerized protein. Preferably, the Env protein contains an introduced glycosylation site. For example, the Zika protein may contain an added N-glycosylation site according to the consensus sequence: Asn-Xaa-Ser/Thr/Cys (where Xaa is selected from genetically encoded amino acids other than Pro (P); that is, Ala (A), Arg (R), Asn (N), Asp (D), Cys (C), Gln (Q), Glu (E), Gly (G), His (H), Ile (I), Leu (L), Lys (K), Met (M), Phe (F), Ser (S), Thr (T), Trp (W), Tyr (Y), and Val (V)). In certain aspects, the introduced glycosylation sequence is Asn-Thr-Thr (NTT). In a particular aspect, the Env protein comprises or consists of SEQ ID NO:7, which has amino acids 291 to 694 of SEQ ID NO: 5, with amino acids DMA at positions 67 to 69 (numbered with respect to SEQ ID NO:8) replaced with amino acids NTT. In certain aspects, the Env protein may comprise of consist of SEQ ID NO:7 with a C-terminal hexahistidine tag.

The polypeptide antigens disclosed herein encompass variations. In certain aspects, the polypeptide may share identity to a disclosed polypeptide. Generally, and unless specifically defined in context of a specifically identified polypeptide, the percentage identity may be at least 90%, at least 95%, at least 97%, or at least 98%. Percentage identity can be calculated using the alignment program Clustal Omega, available at www.ebi.ac.uk/Tools/msa/clustalo/. Sievers et al. “Fast, scalable generation of high-quality protein multiple sequence alignments using Clustal Omega.” (2011 Oct. 11) Molecular systems biology 7:539.

Structural studies on ZIKV Env shows it contains structures and has three distinct domains: a central β-barrel (domain I), an elongated finger-like structure (domain II), and a C-terminal immunoglobulin-like module (domain III). Dai et al., “Structures of the Zika Virus Envelope Protein and Its Complex with a Flavivirus Broadly Protective Antibody,” Cell Host Microbe. 2016 May 11; 19(5):696-704. The central domain I contains around 130 residues in three segments, residues 1-51, 132-192, and 280-295. Residues 147-161 within domain I likely represent a highly flexible loop. The finger-like domain II is formed by two segments, residues 52-131 and residues 193-279. The C-terminal domain III (residues 296-403) displays an IgG-like fold and is contacted by the adjacent E protein monomer. A hydrophobic fusion loop (residues 98-109) is responsible for the membrane fusion between host cell and virus membranes during virus entry, and is highly conserved in flaviviruses.

Suitable E antigens disclosed herein contain one or more, typically all, of domain I, domain II and domain III. In particular aspects, additional amino acids N- or C-terminal to each included domain may also be included. For example, 1, 2, 3, 4, 5, 6, 7, 8, 9, or 10 amino acids may be added. In other aspects, amino acids N- or C-terminal to each included domain may also be deleted. For example, 1, 2, 3, 4, 5, 6, 7, 8, 9, or 10 amino acids may be deleted. Typically, the E antigens disclosed herein contain the conserved epitope, ⁹⁸DRGW¹⁰¹ (SEQ ID NO:6) contained within the fusion loop residues 98-109.

Optionally, the Zika Env polypeptide may contain additional amino acids not from contiguous portions of the Zika Env polypeptide; i.e, they contain a heterologous amino acid portion. Typically, at least one amino acid not found in the wild-type Zika Env protein is contiguous to sequences found in wild-type Zika Env protein. Additions to the protein itself may be for various purposes; for example to facilitation expression or purification. In some aspects, the antigen may be extended at the N-terminus, the C-terminus, or both. In some aspects, the extension is a tag useful for a function, such as purification or detection. In some aspects the tag contains an epitope. For example, the tag may be a polyglutamate tag, a FLAG-tag, a HA-tag, a polyHis-tag (having 4, 5, 6, 7, 8, 9, or 10 contiguous histidines), a Myc-tag, a Glutathione-S-transferase-tag, a Green fluorescent protein-tag, Maltose binding protein-tag, a Thioredoxin-tag, or an Fc-tag. In other aspects, the extension may be an N-terminal signal peptide fused to the protein to enhance expression. While such signal peptides are often cleaved during expression in the cell, some vaccine compositions may contain the Zika antigen with an intact signal peptide. For the purposes of calculating identity to the sequence, additions to the Env protein are not included.

Protease cleavage sites may also be used. During purification, part or all of the cleavage site may be removed. Where only part is removed, a residual portion remains fused to the Zika Env polypeptide. Exemplary protease sites are those cleaved by TEV protease. Alternate protease cleavage site include those sites cleaved by pepsin A, thermoylsin, thrombin, and trypsin. It is understood that cleavage site selection is determined in part by avoiding cleavage or portions of the protein desired to be maintained in the immunogenic formulations.

Typically, such cleavage sites and tags will be present on the C-terminus, but may be present on the N-terminus in different aspects. In certain cases, a cleavage site or tag may be present at both termini. Optionally, any tag or protease site may be fully or partially removed during processing prior to formulating a vaccine composition. For example, the tag and protease cleavage site may be positioned such that protease treatment removes the tag.

Thus, in certain aspects, the antigen administered to the subject may contain contiguous heterologous amino acids fused to the Zika EnvD protein; for example, the administered antigen may contain at least 1 and up to 5, up to 10, up to 20, up to 25, up to 50 or up to 100 contiguous amino acids from a non-Zika source.

In some aspects, the protein may be further truncated. For example, the N-terminus may be truncated by about 10 amino acids, or about 30 amino acids. The C-terminus may be truncated instead of or in addition to the N-terminus. For example, the C-terminus may be truncated by about 10 amino acids, or about 30 amino acids. For purposes of calculating identity to the protein having truncations, identity is measured over the remaining portion of the protein.

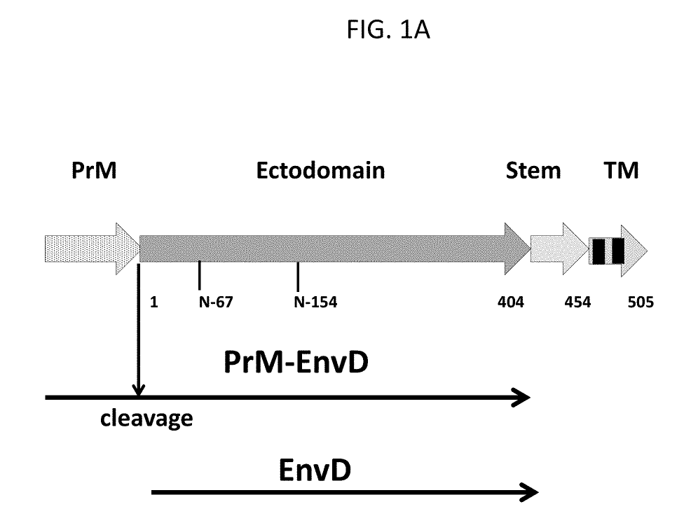

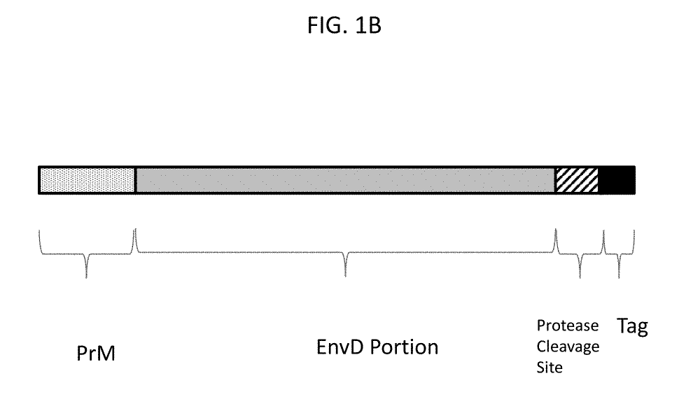

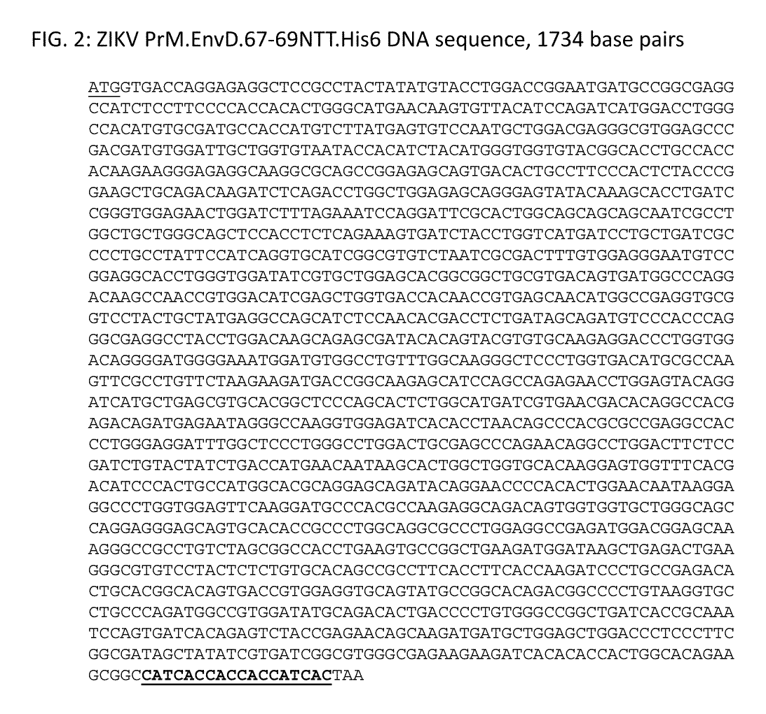

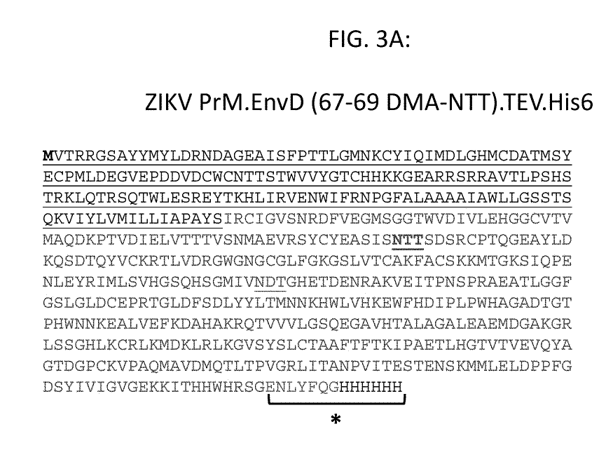

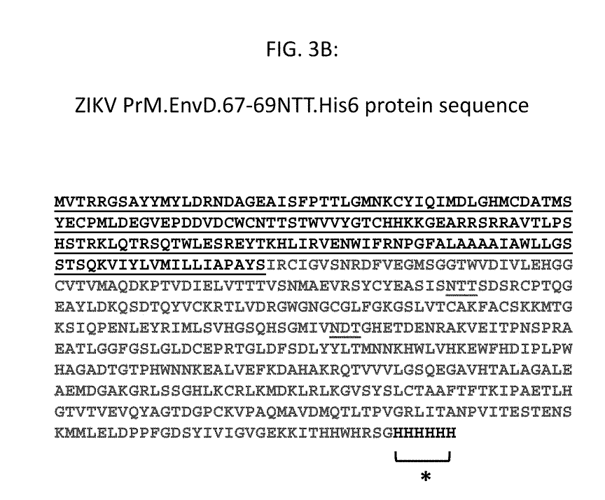

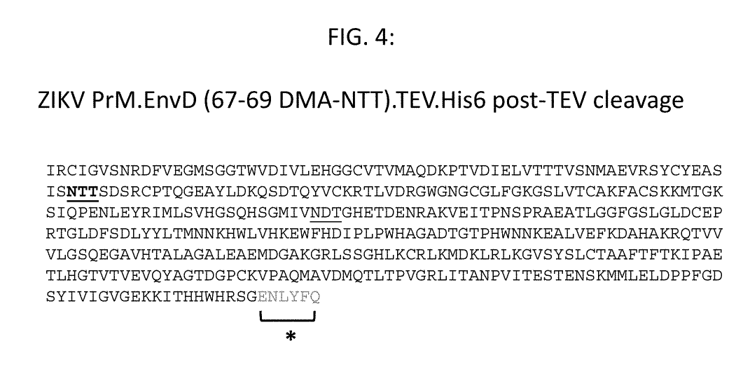

N-terminal additions may be used to enhance protein expression, folding, and/or secretion. Thus, in preferred aspects, the Zika protein may be fused at the N-terminus to “PrM” (i.e both the propeptide and Membrane proteins) shown in FIGS. 1 and 2. The PrM polypeptide may be removed following expression; for example, the PrM domain may be cleaved off in the host cell, e.g., Sf9 cell, by a host cell protease. FIG. 4 provides an example. Other heterologous sequences such as a protease site or tag may also remain attached to the Env protein as administered, with the caveat that protease cleavage sites are not included between the PrM and EnvD portions. Additional portions of the Env protein may be included, but typically are not. Thus, most embodiments will not include the Stem region and will not include the transmembrane region. See FIG. 1.

Exemplary constructs that may be used to produce ZIKV vaccines, as well as the proteins expressed from the constructs, are described in the table below. The BV number refers to the combination of the construct and a particular host cell. Host cells expressing the same construct are indicated in parentheses.

Exemplary Constructs and Proteins for Zika Virus Vaccine

| BV Number | Construct | Precursor | Mature |
|---|---|---|---|
| 1858 | ZIKV EnvD | EnvD | EnvD |
| | | (SEQ ID NO: 7) | (SEQ ID NO: 7) |
| 1865 | ZIKV PrM. EnvD | PrM with EnvD | EnvD |
| | | | (SEQ ID NO: 7) |
| 1903 | ZIKV PrM.EnvD.His6 | PrM EnvD and | EnvD.His6 |
| | | hexahistidine tag | (as SEQ ID NO: |
| | | | 7 with C-term |
| | | | tag) |
| 1944 | ZIKV PrM.EnvD (67-69 | PrM with EnvD, a N- | EnvD.His6 with |
| (2002) | DMA-NTT).His6 | linked glycosylation | NTT introduced |
| | (SEQ ID NO: 3) | site, and hexahistidine | and His6 tag |
| | | tag | (SEQ ID NO: 10) |
| 1993 | ZIKV PrM.EnvD (67-69 | PrM with EnvD, a N- | EnvD TEV |
| | DMA-NTT).TEV.His6 | linked glycosylation | protease- |
| | (SEQ ID NO: 1) | site, and cleavable | cleavable His6 |
| | | histidine tags | with NTT |
| | | (TEV.His6) | introduced |
| | | | (SEQ ID NO: 4) |
| 2009 | ZIKV PrM.EnvD (67-69 | PrM with EnvD, a N- | EnvD with NTT |
| (2037) | DMA-NTT) | linked glycosylation | introduced |
| | | site | (SEQ ID NO: 8) |

### Methods of Manufacturing Zika Virus Protein

Typically, the Zika virus proteins in the compositions are produced by recombinant expression in host cells. Standard recombinant techniques may be used to prepare constructs for expression. For example, the Zika virus proteins can be expressed in insect host cells using a baculovirus system. Examples of insect cells include, but are not limited to Spodoptera frugiperda (Sf) cells (e.g. Sf9, Sf21, Sf22a, which is a rhabdovirus free subclone of Sf9), Trichoplusia ni cells (e.g. High Five cells), and Drosophila S2 cells. In certain aspects, the Sf9 cells are used; for example, BV1944. In other aspects, Sf22a cells are used; for example, BV2002. In embodiments, the baculovirus is a cathepsin-L knock-out baculovirus. In other embodiments, the bacuolovirus is a chitinase knock-out baculovirus. In yet other embodiments, the baculovirus is a double knock-out for both cathepsin-L and chitinase (e.g., BV 2037).

To promote expression and proper folding, chaperone proteins, such as the Hsp40 and Hsc 70 co-chaperones, may be expressed in the host cell. For example, the vector may co-express both the Zika protein, and Hsp40 and Hsc 70 co-chaperones. Alternatively, co-transfection of a vector encoding the Zika antigen and a vector, or vectors, encoding the Hsp40 and Hsc 70 co-chaperones may be performed (e.g., BV2002). Commercial options include ProFold C1 baculovirus DNA which contains chaperone proteins HSC70 and HSP40AB (Vector LLC, San Diego, Calif.)

Typical transfection and cell growth methods can be used to culture the cells. Vectors, e.g., vectors comprising polynucleotides that encode fusion proteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

Purification of the proteins may be performed according to the methods set forth in PCT/US2016/050413, except that preferred methods used herein do not use detergents to extract the protein from the host cell. Rather, the methods disclosed herein use protein secreted into the media, which is purified and then mixed with a non-ionic detergent. Thus, preferably, the EnvD protein used to produce the compositions is EnvD protein secreted into the medium. Purification methods may differ depending on introduction of a glycosylation site. Where a glycosylation site is introduced into the protein, a lectin-based purification step may be used to facilitate purification.

In aspects, the first column may be an ion exchange chromatography resin, such as Fractogel® EMD TMAE (EMD Millipore). In some aspects, the host cell, e.g Sf9 cells, do not completely cleave PrM from EnvD. Uncleaved PrM.EnvD is removed during TMAE flow through column. The second column may be a lentil (Lens culinaris) lectin affinity resin, and the third column may be a cation exchange column such as a Fractogel® EMD SO3 (EMD Millipore) resin. In other aspects, the cation exchange column may be an MMC column or a Nuvia C Prime column (Bio-Rad Laboratories, Inc). Legume lectins are proteins originally identified in plants and found to interact specifically and reversibly with carbohydrate residues. See, for example, Sharon and Lis, “Legume lectins—a large family of homologous proteins,” FASEB J. 1990 November; 4(14):3198-208; Liener, “The Lectins: Properties, Functions, and Applications in Biology and Medicine,” Elsevier, 2012. Suitable lectins include concanavalin A (con A), pea lectin, sainfoin lect, and lentil lectin. Lentil lectin is a preferred column for detergent exchange due to its binding properties. Lectin columns are commercially available; for example, Capto Lentil Lectin, is available from GE Healthcare. In certain aspects, the lentil lectin column may use a recombinant lectin. At the molecular level, it is thought that the carbohydrate moieties bind to the lentil lectin, freeing the amino acids of the protein to coalesce around the detergent resulting in the formation of a detergent core providing nanoparticles having multiple copies of the antigen.

Where a polyhistidine tags are attached to the Zika protein, Ni-NTA columns may be used. Optionally, the lentil lectin column step may be omitted where, for example, the Zika protein does not contain an introduced glycosylation site.

To form nanoparticles, the EnvD protein is eluted from the lentil lectin column using a non-ionic surfactant. The surfactant may be selected from the group consisting of Triton-x-100, PS20, PS40, PS60, and PS65. Preferably, the surfactant is PS20. The surfactant will typically be present in a range of about 0.02% to about 0.05%; about 0.03% PS20 has a good effect on stability of the zika dimers.

The pH of buffers used during extraction and formulation is maintained at pH 7.0 or above. Preferably pH 7.0 to pH 7.6, and more preferably at pH 7.2 to pH 7.5. In particular aspects, the harvest of Zika protein from the cells is performed at pH 7.0 and other steps are performed between pH 7.2 to 7.5.

### Zika Nanoparticle Structure and Function

Early attempts to produce E80 protein were hampered by difficulties during expression and purification. Simply expressing the E80 protein alone resulted in incorrectly folded proteins with poor solubility. This problem was resolved by adding a PrM polypeptide to the N-terminus of the Zika Env protein. Structural analysis of nanoparticles containing sE proteins showed good folding and excellent immunogenicity.

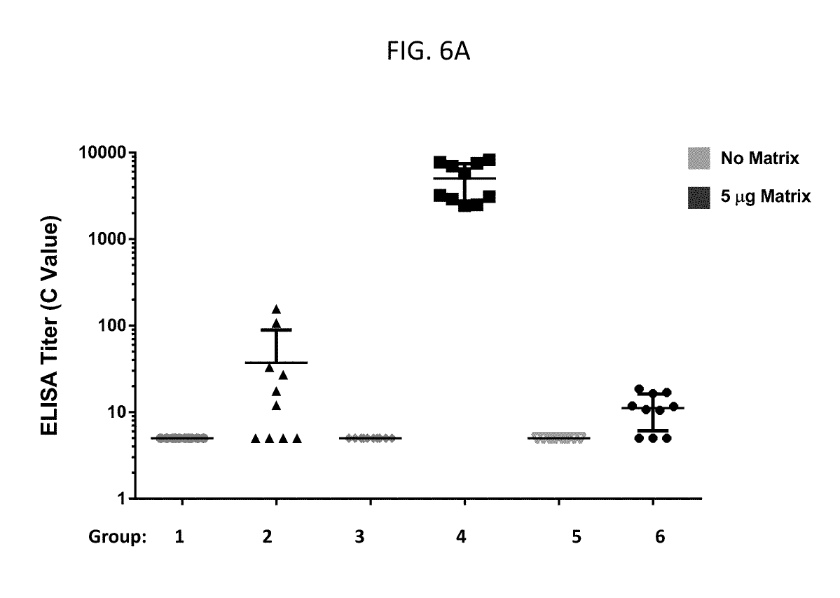

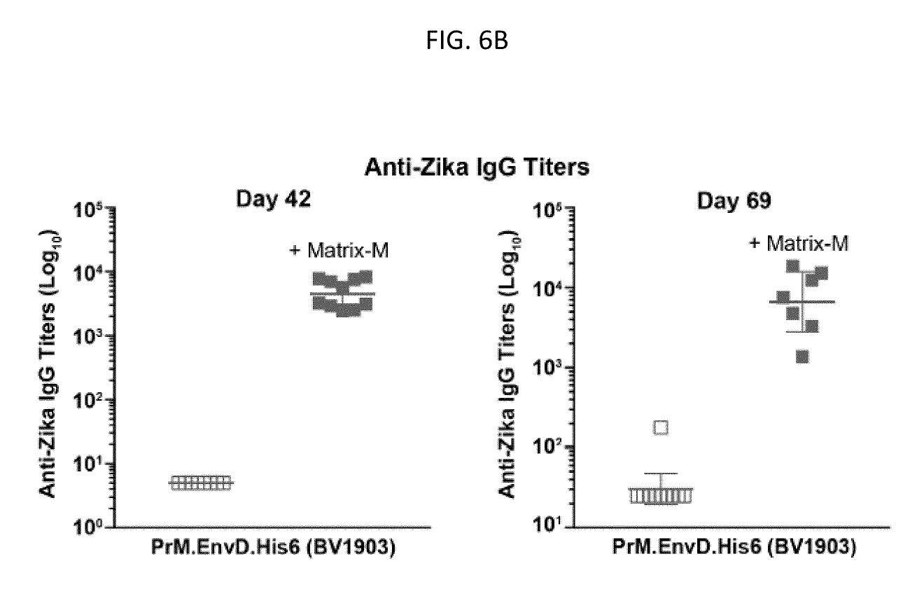

EnvD protein expressed with the PrM portion also gave particularly good immune responses. Administering ZIKV EnvD in combination with 5 μg Matrix-M resulted in antibody titers about 100-fold higher than the HA-1 EnvD ZIKV vaccine and about 500-fold higher than the ZIKV virus iE protein vaccine, likely due to improper folding of this protein. Vaccinating ZIKV EnvD (E80 in FIG. 6A) in combination with 5 μg Matrix-M gave in an antibody neutralization titer response about 50-fold higher than other Zika virus vaccines treated with Matrix-M.

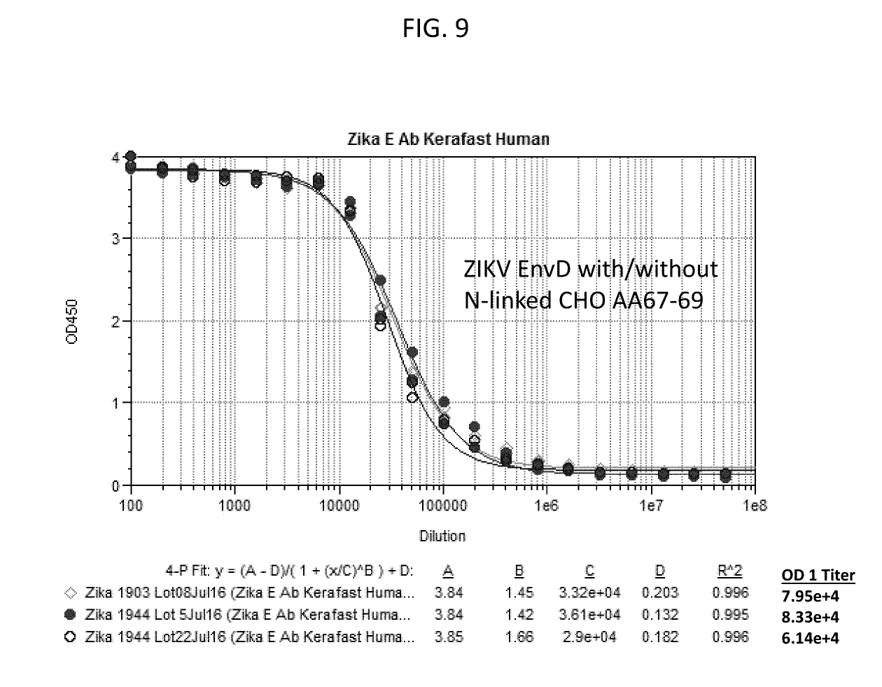

ZIKV EnvD proteins from BV1903 and BV1944 exhibited similar binding kinetics to human convalescent serum (FIG. 9). Thus, the addition of the N-linked glycosylation site does not alter the protein structure or ability to induce immune responses.

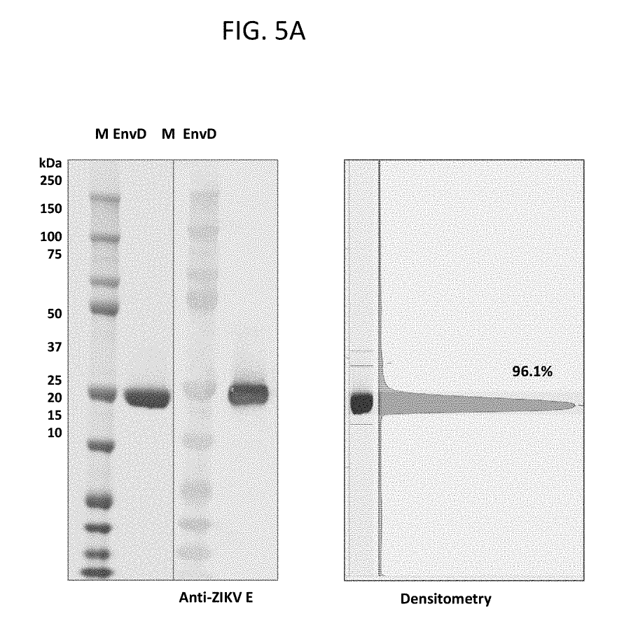

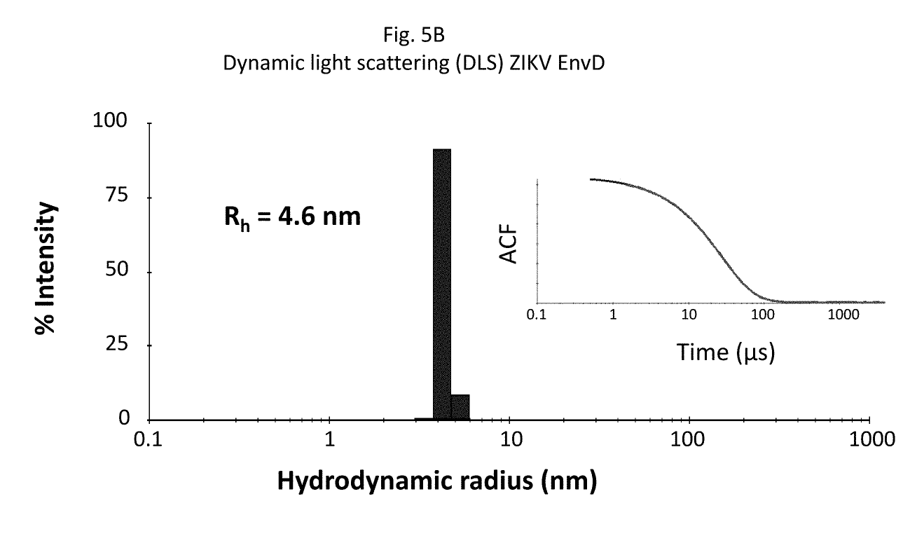

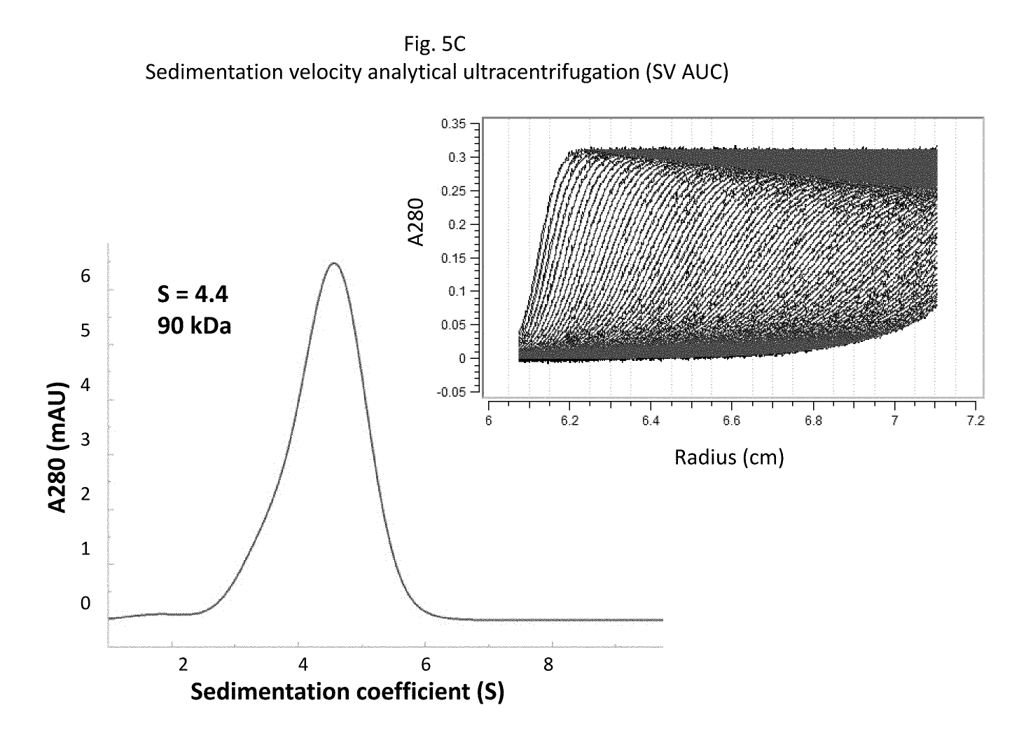

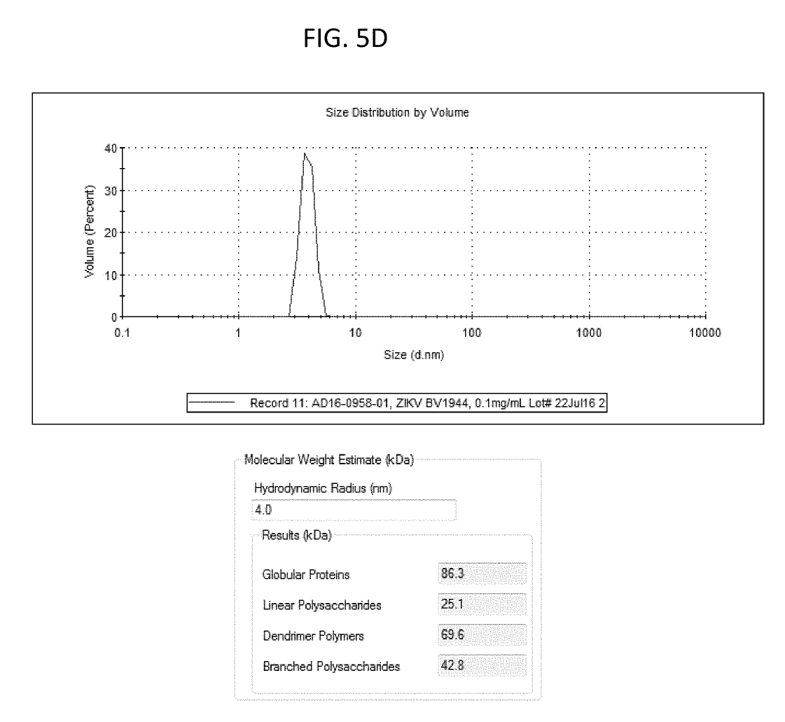

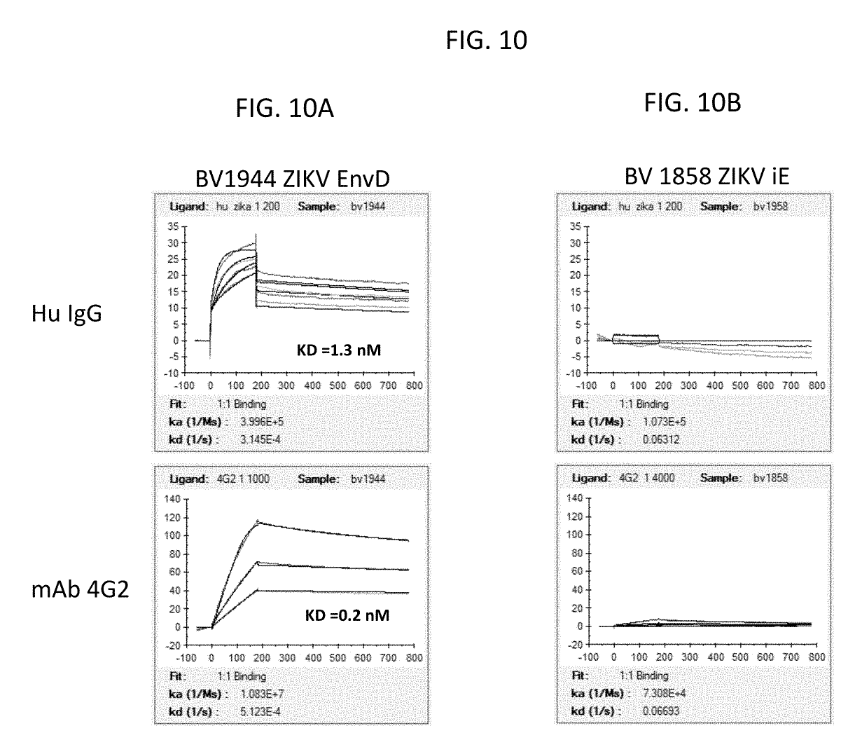

Further analysis of BV1944 ZIKV EnvD showed that the protein forms homodimers, which further assembled into 4 nm to 7 nm structures. FIG. 5. Because transmembrane domains have long been expected to play an important role in higher order structures for proteins, forming dimers and other polymers, our ability to obtain E80 dimers in their absence was unexpected. The BV1944-expressed E protein showed good binding to antibodies known to bind Zika virus proteins. FIG. 10A shows binding to IgG from human convalescent serum and also to the mouse antibody 4G2. In contrast, BV1858-expressed iE protein, which is refolded, did not bind either protein, indicating that refolding does not give rise to correctly-folded protein. FIG. 10B.

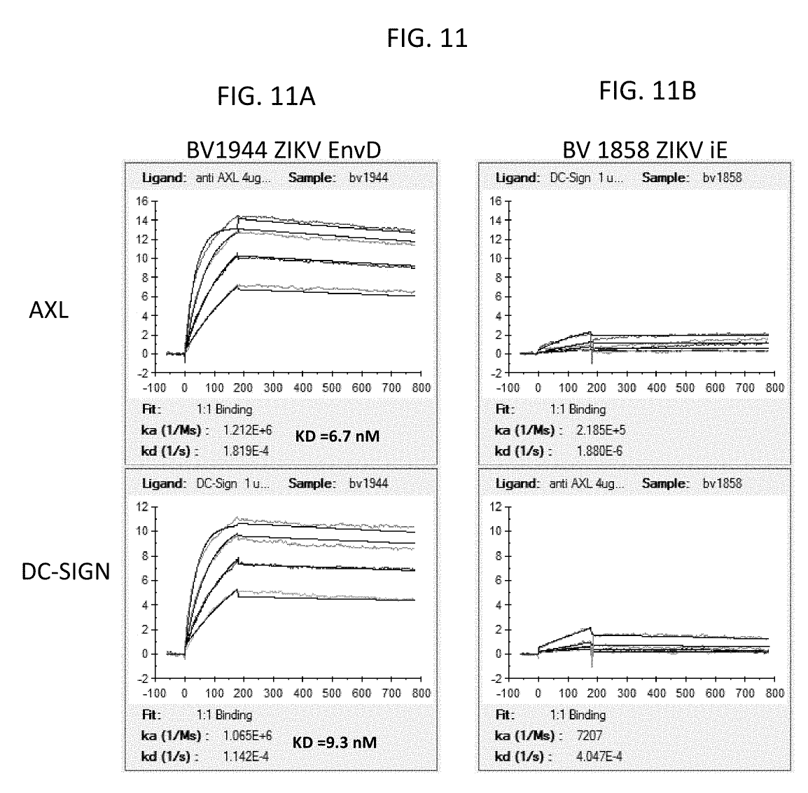

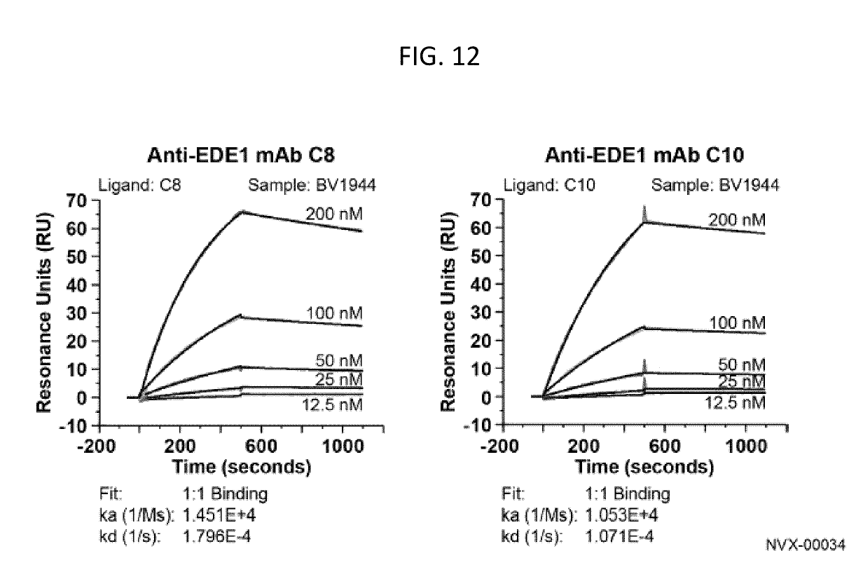

Similar data were obtained for binding to AXL and DC-SIGN, each of which is a known receptor used by Zika to infect cells. FIG. 11. The binding studies showed both proteins bind to the EnvD protein. See FIG. 11A. In contrast, refolded protein (iE) produced from BV1858 did not bind. See FIG. 11B. Dimerisation proved to have beneficial functional effects with respect to immune responses. Zika Virus forms additional epitopes when dimerized. One is referred to EDE-1 (envelope dimer epitope) and, as FIG. 12 shows, two antibodies (Creative Biolabs C8 and C10) that bind specifically to the dimer epitope bind to protein expressed from BV1944.

### Zika Vaccine Formulations

To maintain optimal stability, immunogenic formulations disclosed herein preferably contain both a surfactant and EDTA. The surfactant is typically introduced during a later stage of column purification; for example, a detergent exchange step, and maintained in the formulation used for administering to a subject. The enhanced stability of Zika antigens lacking transmembrane domains obtained with surfactant in the formulation was not expected because detergents have typically been required for stabilising the transmembrane portions of viral proteins, which are often highly hydrophobic.

EDTA was found to promote stability of the formulation and is present in an amount that preserves Zika dimer structures. For example, the EDTA may be present at about 100 μM to about 5 mM, about 500 μM to about 2.5 mM, about 750 μM to about 1.5 mM, or about 1 mM.

NaCl may be present in the composition. For example, the NaCl may be present at about 100 mM to about 800 mM, 200 mM to about 600 mM, 250 mM to about 400 mM, or about 300 mM.

To maintain pH above 7.0, a particularly suitable buffer is a NaPO₄ buffer. Such a buffer can be obtained, for example, by mixing 1 M NaH₂PO₄ (monobasic) and 1 M Na H₂PO₄ (dibasic) stock solutions. The NaPO₄ may be present in a formulation at about 10 mM to about 50 mM, about 20 mM to 40 mM, or, preferably about 25 mM. Thus, suitable formulations may contain about 20 to about 40 mM NaPO4, pH 7.2 to 7.6, about 200 mM to about 400 mM NaCl, 0.02% to 0.05% surfactant, and about 750 μM to about 1.5 mM EDTA. A preferred zika composition contains about 25 mM NaPO4, pH 7.5, about 300 mM NaCl, about 0.03% PS20, and about 1 mM EDTA.

In certain aspects, the zika formulations may be provided in kit form, optionally along with instructions. For example, the kit may contain the zika protein in a formulation alone or with adjuvant. Advantageously, as disclosed herein, Matrix M1 can be combined with zika without loss of protein or dimer stability. Such a combination enhances administration and hence such pre-mixed vaccines are advantageous.

### Immune Responses

The present disclosure provides methods that can induce protective immune responses. In some aspects, the protective immune responses can increase neutralizing antibody levels when administering Zika virus vaccine with an adjuvant as described herein. In a preferred aspect, the protective immune responses are induced by Zika antigen when administered with a Matrix-M adjuvant. The immune responses obtained by compositions include neutralizing antibodies. In particular aspects, the response includes antibodies against epitopes present only in dimers of Zika Env proteins. Thus in particular aspects, the immunogenic compositions comprise Env dimers that contain at least one dimer epitope absent from the equivalent Env monomer; for example, probing the monomer preparation of Env and a dimer preparation of Env by antibody (e.g. western blot) detects the dimer but not the monomer. In certain other aspects, antibodies may be produced and purified to use for passive administration to treat zika infection. Such antibodies may be produced as monoclonal antibodies. In other aspects, they may be produced as polyclonal antibodies; for example in transgenic animals such as transgenic bovines. Exemplary transgenic bovines include transchromasomal (Tc) bovine, which are triple knockouts in the endogenous bovine immunoglobulin genes (IGHM−/− IGHML1−/− IGL−/−) and carry a human artificial chromosome vector labeled as isKcHACD. See Sano et al. Physiological level production of antigen-specific human immunoglobulin in cloned transchromosomic cattle. PloS one 2013; 8:e78119; Hooper et al DNA vaccine-derived human IgG produced in transchromosomal bovines protect in lethal models of hantavirus pulmonary syndrome. Science translational medicine 2014; 6:264ra162; Matsushita et al Triple immunoglobulin gene knockout transchromosomic cattle: bovine lambda cluster deletion and its effect on fully human polyclonal antibody production. PloS one 2014; 9:e90383; Dye et al Production of Potent Fully Human Polyclonal Antibodies against Ebola Zaire Virus in Transchromosomal Cattle. Scientific reports 2016; 6:24897.

### Adjuvants

In certain aspects, the compositions disclosed herein may be combined with one or more adjuvants to enhance an immune response. In particular aspects, the compositions are prepared without adjuvants, and are thus available to be administered as adjuvant-free compositions.

### Mineral-Based Adjuvants

In some aspects, the adjuvant can be aluminum phosphate, aluminum hydroxide, aluminum, or calcium phosphate. In some aspects, the aluminum may be AlP0₄ or Al(OH)₃. The amount of aluminum is present per dose is typically in a range between about 400 μg to about 1250 μg. For example, the aluminum be present in a per dose amount of about 300 μg to about 900 μg, about 400 μg to about 800 μg, about 500 μg to about 700 μg, about 400 μg to about 600 μg, or about 400 μg to about 500 μg. Typically, the aluminum is present at about 400 μg for a dose of 120 μg of vaccine formulation.

### Bacterial Adjuvants

In some aspects, the adjuvant in the vaccine compositions can be a bacterial adjuvant. The bacterial adjuvant can be obtained from mycobacterial species, mycobacterial components such as monophosphoryl lipid A, trehalose dimycolate, muramyl dipeptide, corynebacterium species, B. pertussis, or lipopolysaccharide. In some aspects, the adjuvant in the vaccine compositions can be any bacterial adjuvant that is suitable for vaccine compositions.

### Oil-Based Adjuvants

Further, in some aspects, the adjuvant in the vaccine formulations can be an oil-based emulsion. In other aspects, the oil-based emulsion can be saponins, starch oil, or Freund's complete or incomplete adjuvant. In some aspects, the adjuvant in the vaccine formulations can be any oil-based emulsion that is suitable for vaccine formulations.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (lsconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structure

In some aspects, saponin-based adjuvants can be formulated in immune stimulating complex (ISCOM). In other aspects, saponin-based adjuvants can be formulated in ISCOM-Matrix structures. Saponin fractions may be administered in the form of a cage-like particle referred to as an ISCOM (Immune Stimulating COMplex). ISCOMs may be prepared as described in EP0109942B1, EP0242380B1 and EP0180546 B1. In particular aspects a transport and/or a passenger antigen may be used, as described in EP 9600647-3 (PCT/SE97/00289).

### Matrix Adjuvants

In some aspects, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid, often phoshatidylcholine. The ISCOM matrix complexes may also contain one or more other immunomodulatory (adjuvant-active) substances, not necessarily a glycoside, and may be produced as described in EP0436620B1.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

According to one aspect, the saponin fraction integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770.

In particular aspects, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In another aspect, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In yet another aspect, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions.

In other aspects, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In some aspects, the remaining portion is one or more ISCOM matrix or complexes where each matrix or complex particle contains only one saponin fraction. In other aspects, the ISCOM matrix or complex particles may contain more than one saponin fraction.

In particular compositions, the saponin fraction in a first ISCOM matrix or ISCOM complex particle is Fraction A and the saponin fraction in a second ISCOM matrix or ISCOM complex particle is Fraction C.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference for those disclosures

In a preferred aspect, the saponin-based adjuvant is a Matrix-M™ adjuvant. In some aspects, the Matrix-M™ adjuvant can be extracted from the Quillaja saponaria Molina tree. In some aspects, the adjuvant can be formulated and purified with cholesterol and phospholipid. In other aspects, Matrix-M™ adjuvant can consist of two populations of individually formed particles. These two particles may have complementary properties. In some aspects, the particles can be about 25-55 nm, about 30-50 nm, or about 35-45 nm. In a preferred aspect, the particle is 40 nm.

In some aspects, one particle of the Matrix-M™ can be Fraction-A (Matrix-A) and the other particle can be Fraction-C (Matrix-C). In some aspects, Matrix-M™ can include optimal ratios of Matrix-A and Matrix-C components to maintain high-adjuvant activity with optimal safety margin. For example, Matrix-M™ comprises 85% Matrix-A and 15% Matrix-C, referred to as Matrix-M1™. In other aspects, the Matrix-M™ comprises 92% Matrix-A and 8% Matrix-C, referred to as Matrix-M2™. Unless specified otherwise, the Matrix-M™ used throughout the disclosure is Matrix-M1™

In some aspects, the administration dose of Matrix-M™ adjuvant can be about 1 to about 100 μg, about 5 to about 95 μg, about 10 to about 90 μg, about 15 to about 85 μg, about 20 to about 80 μg, about 25 to about 75 μg, about 30 to about 70 μg, about 35 to about 65 μg, about 40 to about 60 μg, about 45 to about 55 μg, about 50 μg, or any values in between.

Without wishing to be bound by theory, Matrix-M adjuvant can induce high and long-lasting levels of broadly reacting antibodies supported by a balanced TH1 and TH2 type of response, including biologically active antibody isotypes such as murine IgG2a, multifunctional T cells and cytotoxic T lymphocytes. Generally, Matrix-M adjuvant can enhance immune response and promote rapid and profound effects on cellular drainage to local lymph nodes creating a milieu of activated cells including T cells, B cells, natural killer cells, neutrophils, monocytes, and dendritic cells. In part, Matrix-M™ can enhance the combination of antibody and cellular immune response, whereas most oil emulsion-based adjuvants mainly promote antibody responses.

### Synthetic and Other Adjuvants

In some aspects, the adjuvant in the vaccine formulations can be a synthetic adjuvant. In other aspects, the synthetic adjuvant can be analogues of muramyl peptide, or synthetic lipid A. In some aspects, the adjuvant in the vaccine compositions can be any synthetic adjuvant that is suitable for vaccine compositions. In some aspects, compositions other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/Tween® 80 emulsion. In some aspects, the adjuvant may be a paucilamellar lipid vesicle; for example, Novasomes®. Novasomes® are paucilamellar nonphospholipid vesicles ranging from about 100 nm to about 500 nm. They comprise Brij 72, cholesterol, oleic acid and squalene. Novasomes have been shown to be an effective adjuvant (see, U.S. Pat. Nos. 5,629,021, 6,387,373, and 4,911,928

### Administration and Dosage

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

Compositions may be administered on a single dose schedule or a multiple dose schedule. Multiple doses may be used in a primary immunization schedule or in a booster immunization schedule. In a multiple dose schedule the various doses may be given by the same or different routes e.g., a parenteral prime and mucosal boost, a mucosal prime and parenteral boost, etc. In some aspects, a follow-on boost dose is administered; for example, about 2 weeks, about 3 weeks, about 4 weeks, about 5 weeks, or about 6 weeks after the prior dose. In view of Zika's looming establishment as a persistent threat, a consolidating booster may be considered. Consolidating booster may administered at about 28 weeks.

The disclosed vaccine compositions are administered to a subject to induce an immune response. As used herein, “subject” refers to mammalian subjects (e.g. canine, feline, equine, bovine, ungulate etc.) for whom vaccination is desired. Typically, the subject is a human. For example, a human female that is or intending to become pregnant in the near future.

### EXAMPLES

### Example 1

### Expression and Purification of ZIKV Proteins

Zika virus envelope dimer (EnvD) vaccine nanoparticle based on the Zika virus (ZIKV) Brazilian strain ZikaSPH2015 polyprotein sequence [Genbank accession number ALU33341.1]. The ZIKV polyprotein amino acid (AA) 125 to 215 is the propeptide (Pr), AA 216-290 is the membrane protein (M), and AA 291-795 is the full length envelope protein (Env). AA 291-694 are approximately 80% of the N-terminus of the Env ectodomain (E80) and are the amino acids that define ZIKV Env dimers (EnvD). The EnvD sequence was further mutated to include NTT at positions 67 to 69 and introduced in to pNvax3765, which was cotransfected into Sf9 cells with ProFold C1 baculovirus DNA with Hsc70/Hsp40 chaperone (AB Vector LLC, San Diego, Calif.) to make BV1993. In an alternate Example, a poly-His tag was added to the C-terminus of EnvD to obtain BC1944. In a further alternate Example, the EnvD sequence lacking the NTT was introduced into baculovirus to make BV1903.

Constructs for the Production of Zika Virus Vaccine are shown below.

| BV Number | Construct | Precursor | Mature |
|---|---|---|---|
| 1858 | ZIKV EnvD | EnvD | EnvD |
| | | (SEQ ID NO: 7) | (SEQ ID NO: 7) |
| 1865 | ZIKV PrM. EnvD | PrM with EnvD | EnvD |
| | | | (SEQ ID NO: 7) |
| 1903 | ZIKV PrM.EnvD.His6 | PrM EnvD and | EnvD.His6 |
| | | hexahistidine tag | (as SEQ ID NO: 7 with |
| | | | C-term tag) |
| 1944 | ZIKV PrM.EnvD (67-69 | PrM with EnvD, a N- | EnvD.His6 with NTT |
| (2002) | DMA-NTT).His6 | linked glycosylation | introduced and His6 tag |
| | (SEQ ID NO: 3) | site, and hexahistidine | (SEQ ID NO: 10) |
| | | tag | |
| 1993 | ZIKV PrM.EnvD (67-69 | PrM with EnvD, a N- | EnvD TEV protease- |
| | DMA-NTT).TEV.His6 | linked glycosylation | cleavable His6 with |
| | (SEQ ID NO: 1) | site, and cleavable | NTT introduced |
| | | histidine tags | (SEQ ID NO: 4) |
| | | (TEV.His6) | |
| 2009 | ZIKV PrM.EnvD (67-69 | PrM with EnvD, a N- | EnvD with NTT |
| (2037) | DMA-NTT) | linked glycosylation site | introduced |
| | | | (SEQ ID NO: 8) |

BV2002 and BV1944 express a Zika Antigen having the same sequence and differ in the insect host cell strain used. BV1944 was made in a rhabdovirus-free sub-clone of Sf9 cells, referred to as Sf22a, whereas BV2002 was made in Sf9 cells.

Purification of each EnvD protein was dependent on the protein. After harvest, initial purification was by TMAE IEX column followed by capto lentil lectin, where the introduced glycosylation step was present, and with an optional Ni-NTA step, where a poly-His tag was present.

Production of the ZIKV EnvD antigen was initiated by infecting Sf9 cells in exponential growth with the recombinant baculovirus described above. The infected culture was harvested through a depth filter and collected in product tanks. Leupeptin was added to the harvested product, which was diluted and pH adjusted. The product was then purified by flowing through from a Fractogel TMAE anion exchange column to remove host cell proteins and nucleic acids. The flow through product was bound to a Capto Lentil Lectin affinity column to remove non-glycosylated impurities and the produce was eluted with Methyl α-D-mannopyranoside. The resulting product was then bound to a nickel sepharose IMAC column and eluted with imidazole. This step removed non-His-tagged impurities.

After removal of non-His-tagged impurities, we performed nano filtration to remove viruses and a Tangential Flow Filtration (TFF) step to remove residual imidazole and nickel from the IMAC column product. Finally we performed buffer exchange into the final formulation buffer at the desired concentration. The product was then filtered through a sterilizing filter (0.20 μm) to produce the bulk drug substance. Bulk drug substance was clear and colorless. EnvD proteins from clones BV2009 and BV2037 are prepared in the same way. The express the same protein but differ in the host strain used.

### Example 2

### Stimulation of Immune Response in Mice

Protein from BV1903 was purified according to Example 1, except without lentil lectin in view of the BV1903 protein lacking an introduced glycosylation site. To test if the Zika virus secreted envelope protein produced from BV1903 construct, can result in an improved immune response in vivo, various Zika virus vaccines with either the absence or presence of Matrix-M™ adjuvant were tested. Unless otherwise specified, Matrix-M1 was used throughout. The experimental design is shown in Table 2 below. In brief, sixty mice were randomized to six experimental groups (N=10/group). Groups 1 and 2 were administered with BV1878 Zika virus HA1-sE fusion protein while Group 2 also received a dose of Matrix-M™ adjuvant. Groups 3 and 4 were administered with BV1903-expressed EnvD while Group 4 also received a dose of Matrix-M™ adjuvant. Groups 5 and 6 were administered with BV1858 ZIKV iE refolded protein while Group 6 also received a dose of Matrix-M™ adjuvant.

All groups were immunized with their respective vaccines either with or without Matrix-M™ adjuvant on Day 0, 28, and 56. Blood was drawn and processed according to the protocols known in the art on Day 1, 42, and 84 to examine the immune response by performing ELISA antibody titer assays, microneutralizing antibody titer assays, and Zika virus protein receptor binding assays. BV1878 ZIKV HA-1sE (hemagglutinin-1 secreted envelope protein) contains the EnvD sequence without the NTT and contains an N terminal HA-1 sequence to promote secretion. It does not include the PrM N-terminal portion and was produced using a ProFold™ C1 vector (AB Vector LLC, San Diego, Calif.). BV1903 ZIKV virus sE was a PrM.EnvD His6 Zika virus. BV1858 ZIKV virus iE was an EnvD Zika virus protein produced without PrM domains, which did not fold correctly and was re-folded in vitro.

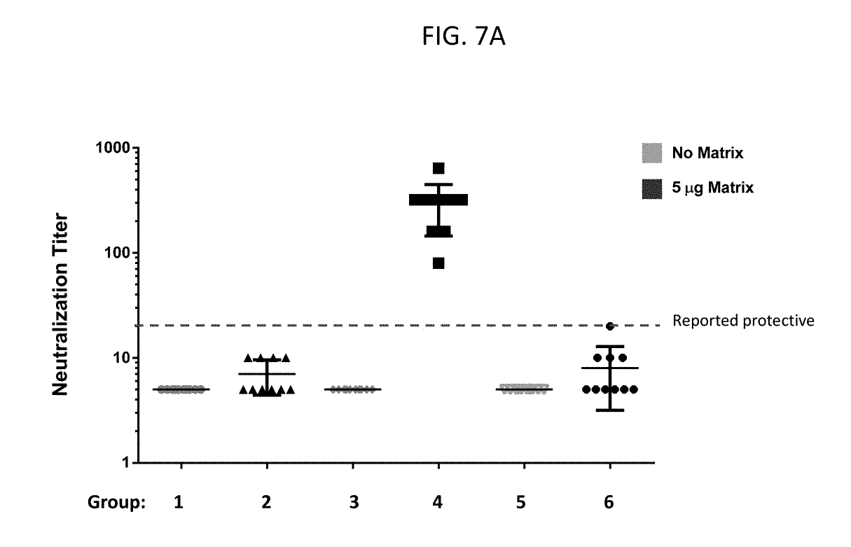

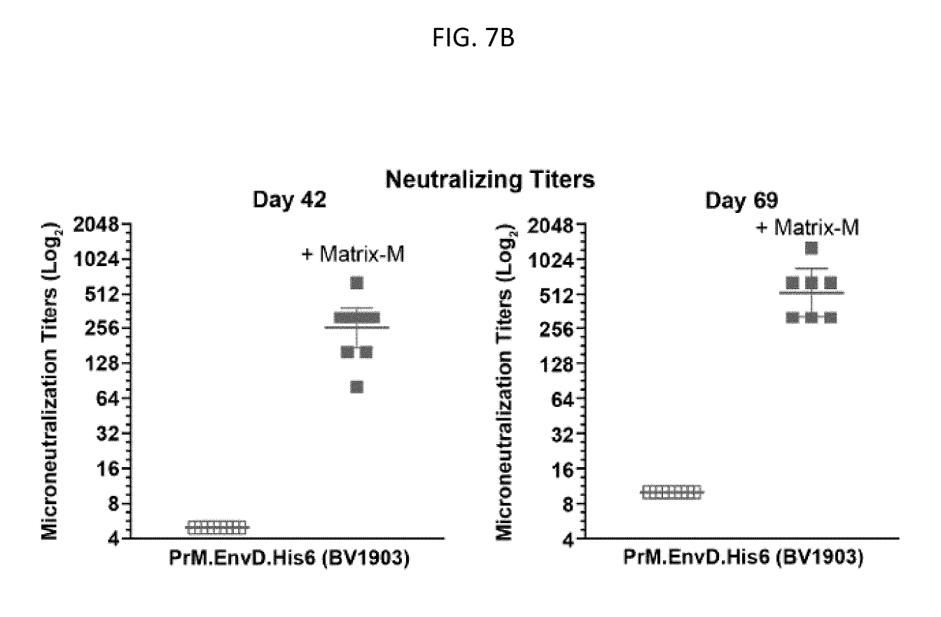

As illustrated in FIG. 6, mice treated with both ZIKV sE secreted protein vaccine and Matrix-M™ adjuvant resulted in the highest ELISA antibody titer response by Day 42 compared to other Zika virus vaccines that were also in combination with Matrix-M™ (about 100-fold higher than the HA-1sE ZIKV vaccine and about 500-fold higher than the ZIKV virus iE protein vaccine). Mice treated with both ZIKV virus sE secreted protein vaccine and Matrix-M™adjuvant resulted in an antibody neutralization titer response that was about 50-fold higher than other Zika virus vaccines treated with Matrix-M™ by Day 42 (FIG. 7). FIGS. 6B and 7B show that the immune responses were maintained for extended periods.

TABLE 2
Various Zika Virus Vaccines with Matrix-M ™ Adjuvant

| | | | | Immunization | Blood |
|---|---|---|---|---|---|
| Group | Number | Vaccine | Matrix-M ™ | Date | Draws |
| 1 | 10 | BV1878 | None | 0, 28, 56 | (−)1, 42, |
| 2 | 10 | 10 μg ZIKV HA1-sE | 5 μg | | and 84 |
| | | (HA1-sE fusion) | | | |
| 3 | 10 | BV1903 | None | 0, 28, 56 | (−)1, 42, |
| 4 | 10 | 5 μg ZIKV PrM | 5 μg | | and 84 |
| | | EnvD (secreted) | | | |
| 5 | 10 | BV1858 | None | 0, 28, 56 | (−)1, 42, |
| 6 | 10 | 5 μg ZIKV iE | 5 μg | | and 84 |
| | | (refolded) | | | |

Abbreviations: ZIKV: Zika virus; HA: hemagglutinin; sE: secreted envelope protein; envelope protein.

### Example 3

### Analysis of Protein Binding

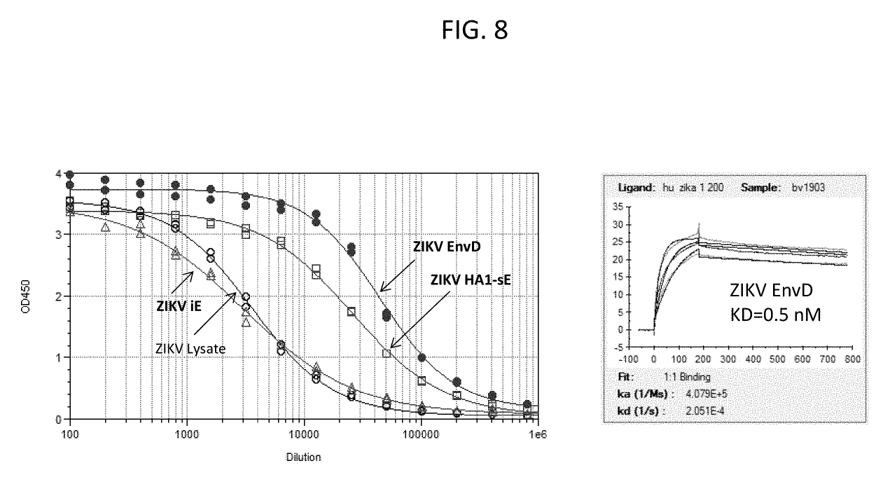

Zika vaccines proteins prepared in accordance with Example 1 were tested with respect to binding to antibodies and to other proteins involved in Zika infection. The data established that protein expression was better when the EnvD protein was produced using the N-terminal PrM extension as used in BV1903 and BV1944. The refolded protein, from BV1858, and the secreted protein with influenza HA1 N-terminus, from BV1878, were less structurally sound. Indeed, the refolded protein was especially poor. See FIG. 8.

Introduction of the glycosylation site into the BV1903-encoded protein, to provide the BV9144-encoded protein, facilitates protein purification on lentil lectin columns. However, it was important to confirm that the mutation did not prevent antibody binding to the protein. FIG. 9 compares binding to the proteins with and without the introduced site and confirms binding of convalescent serum binds well to each. Further analysis used a biosensor approach to determine the ability of proteins to bind to anti-Zika antibody and to proteins that native Zika virus binds to. FIG. 10A confirms that EnvD from BV1944 binds to two antibodies, IgG from a human infected with Zika, and mAB 4G2. See Nawa et al., “Development of dengue IgM-capture enzyme-linked immunosorbent assay with higher sensitivity using monoclonal detection antibody,” J Virol Methods. 2001 March; 92(1):65-70. In contrast, refolded insoluble protein from BV1858 did not exhibit binding. See FIG. 10B.

FIGS. 11A and 11B compares biosensor experiments that demonstrate binding of the EnvD from BV1944, but not refolded protein from BV1858 to AXL, a candidate Zika receptor (Miner et al. “Understanding How Zika Virus Enters and Infects Neural Target Cells,” Cell Stem Cell, Volume 18, Issue 5, 559-560) and DC-SIGN (Hamel et al., “Biology of Zika Virus Infection in Human Skin Cells,” J Virol. 2015 September; 89(17):8880-96). These data establish that expression of the protein with the N-terminal PrM portions provides for expression of correctly folded protein.

FIG. 12 shows binding of anti-EDE1 antibodies to ZIKV protein from BV1944. Binding curves were obtained by passing different concentration, as indicated, over biosensor chips on which the anti-EDE1 mAb C8 (left panel) or anti-EDE1 mAb C10 (right panel) were immobilized. Kinetic values were obtained by fitting the association and dissociation responses to a 1:1 binding model. This epitope bridges two envelope protein subunits on the Zika virus surface and has broadly neutralizing activity, making it an especially beneficial epitope for inducing an immune response.

### Example 4

### ZIKV EnvD Induces Neutralizing Antibodies in Mice

BV1944 EnvD was purified as described in Example 1 and administered to mice as shown in the table below:

| | | BV1944 | | | Immunization | |
|---|---|---|---|---|---|---|
| Group | N/Group | EnvD | AlOH | Matrix M | Day | Blood Draws |
| 1 | 10 | 5 μg | 0 | 0 | 0, 21 | −1, 20, 46 |
| 2 | 10 | 5 μg | 50 μg | 0 | 0, 21 | −1, 20, 46 |
| 3 | 10 | 5 μg | 0 | 5 μg | 0, 21 | −1, 20, 46 |

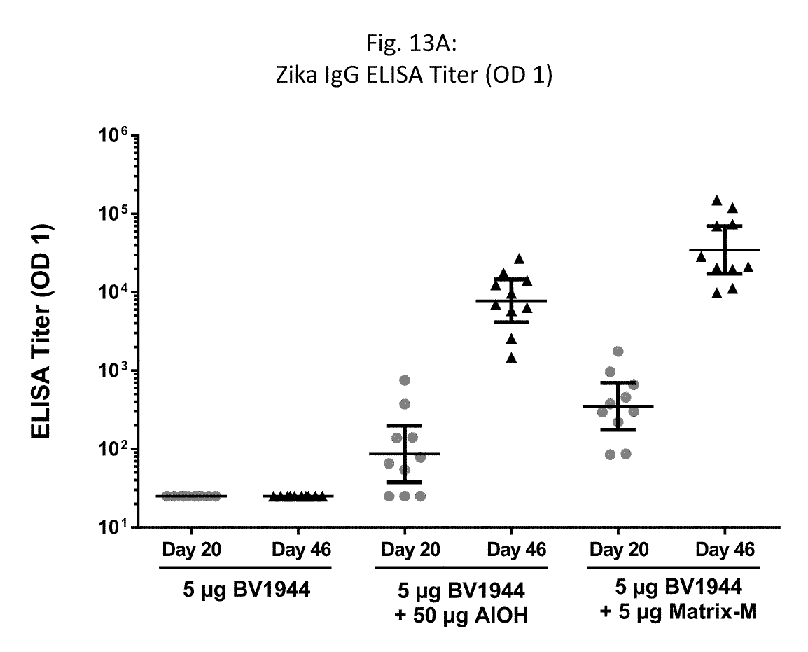

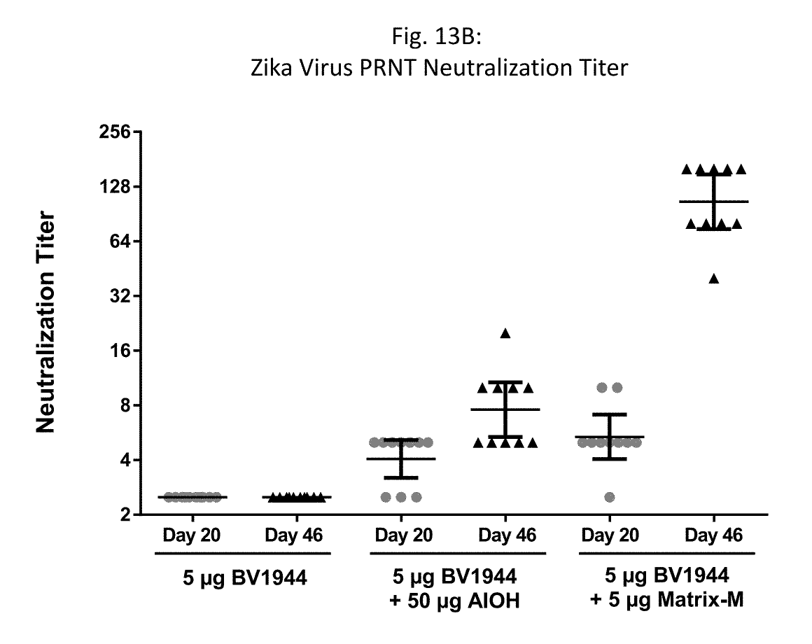

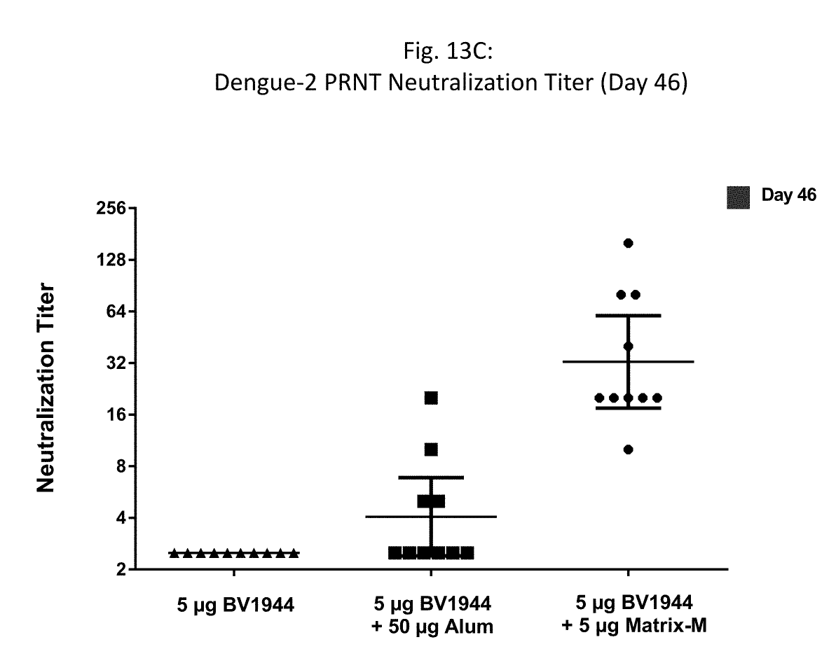

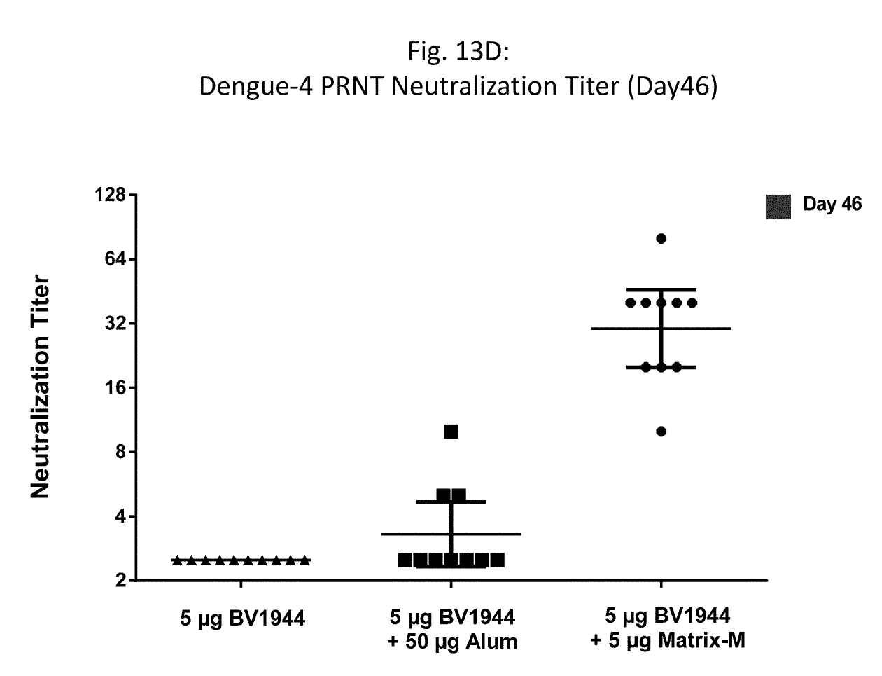

FIG. 13A shows a time-course of the ELISA titer responses at 20 and 46 days. High titers were obtained with either AlOH or with Matrix-M adjuvant. Plaque-reduction neutralization tests (“PRNT”) against Zika were performed to identify neutralizing antibodies. (See World Health Organization Department of Immunization Vaccines Biologicals. 2007. Guidelines for plaque-reduction neutralization testing of human antibodies to dengue viruses. World Health Organization, Geneva, Switzerland. WHO/IVB/07.07) FIG. 13B shows the results for Groups 1-3. By day 20 a response was observed for both adjuvanted groups, Groups 2 and 3. At day 46 both Groups showed elevated neutralizing antibodies.

Cross neutralization against two strains of Dengue (DENV-2 and DENV-4) were also demonstrated. FIG. 13C shows the ZIKV EnvD induces neutralization antibodies against Dengue-2. FIG. 13D shows the ZIKV EnvD induces neutralization antibodies against Dengue-4. For both DENV-2 and DENV-4 Matrix-M1 showed substantially greater production of neutralizing antibodies.

### Example 5

### ZIKV EnvD Induces a Protective Immune Response

The nonhuman primate study was initiated using EnvD from BV2002. Five groups of Rhesus macaques (n=4) were treated as shown in the table below:

| | | | | | | | | Challenge | Serum |
|---|---|---|---|---|---|---|---|---|---|
| | | | | Aluminum | | Immunization | Blood | Study | viral |
| Group | Vaccine | Matrix-M | | hydroxide | | Day | Draw | Day 56 | loads |
| 1 | PBS-Control | 0 | μg | 0 | μg | 1, 29 | 0, 28, 42, | ZIKV-BR | 56-64 |
| | | | | | | | 56 | | |
| 2 | 50 μg | 0 | μg | 0 | μg | 1, 29 | 0, 28, 42, | ZIKV-BR | 56-64 |
| | ZIKV | | | | | | 56 | | |
| | EnvD | | | | | | | | |
| 3 | 5 μg | 50 | μg | 0 | μg | 1, 29 | 0, 28, 42, | ZIKV-BR | 56-64 |
| | ZIKV | | | | | | 56 | | |
| | EnvD | | | | | | | | |
| 4 | 25 μg | 50 | μg | 0 | μg | 1, 29 | 0, 28, 42, | ZIKV-BR | 56-64 |
| | ZIKV | | | | | | 56 | | |
| | EnvD | | | | | | | | |
| 5 | 50 μg | 0 | μg | 800 | μg | 1, 29 | 0, 28, 42, | ZIKV-BR | 56-64 |
| | ZIKV | | | | | | 56 | | |
| | EnvD | | | | | | | | |

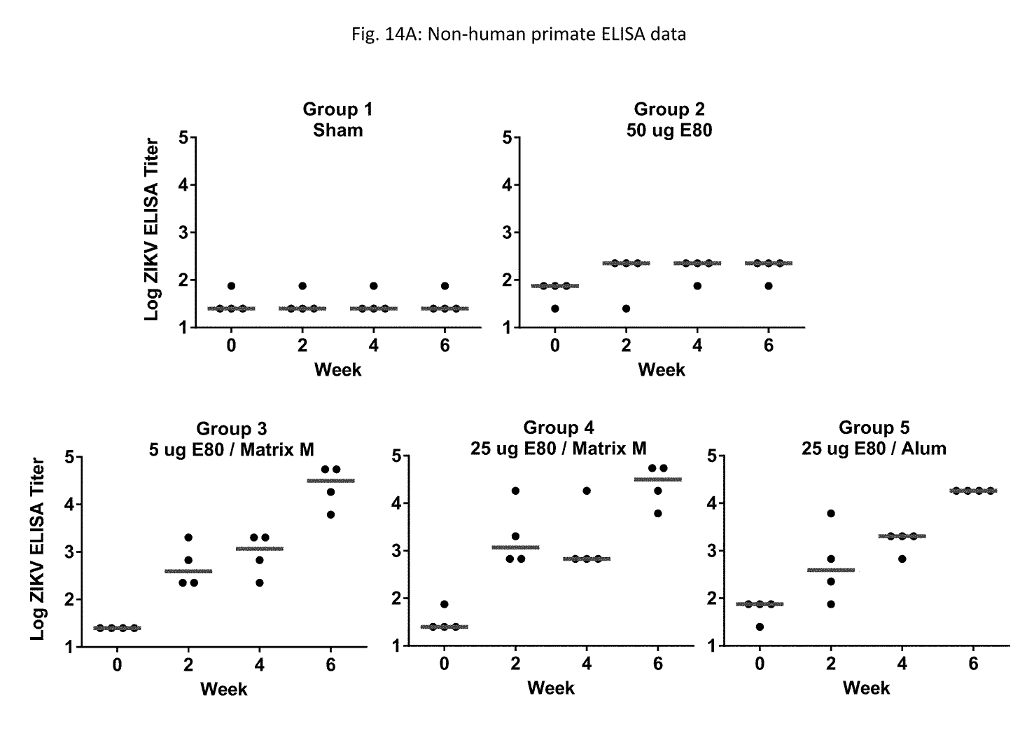

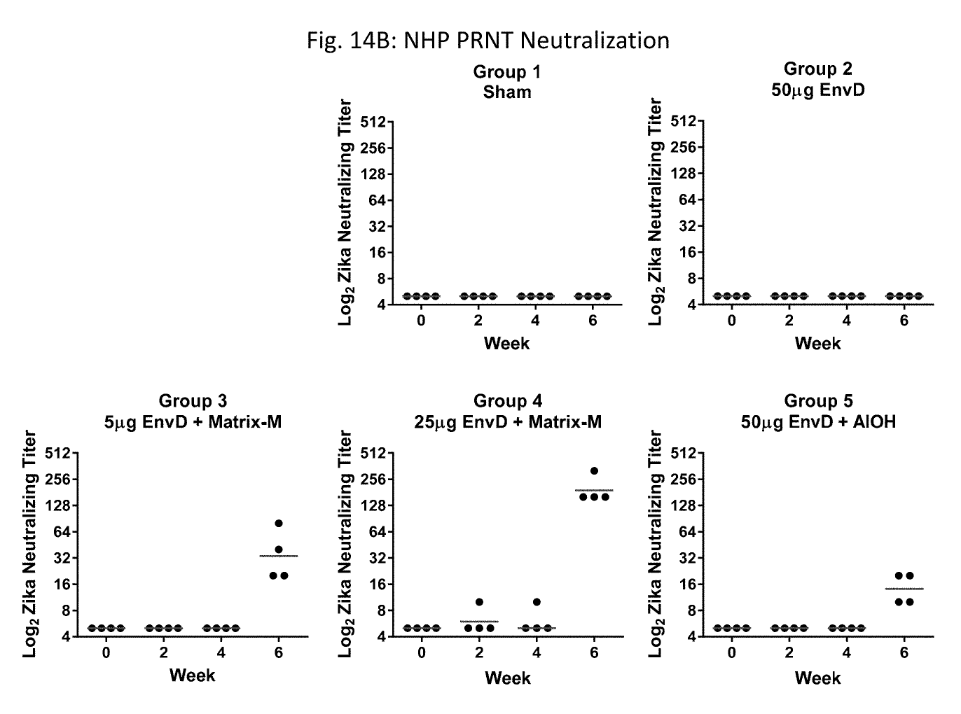

The Rhesus macaques induced excellent ELISA binding and neutralizing antibody (NAb) responses. FIG. 14A shows ELISA data for each of the five groups. All groups but Group 1 shows a response with the most pronounced responses in the adjuvanted groups. The dose-sparing effect of Matrix M was remarkably pronounced. Neutralization data using the PRNT assay is shown in FIG. 14B. For the PRNT experiments, a neutralization titer of 20 is considered protective in monkey challenge studies with Zika virus. Groups 3 and 4 exceeded this neutralization titer by week 6. These data establish excellent immunogenicity and protective efficacy of Matrix-M1-adjuvanted EnvD compositions.

### Example 6

### Stable Zika Formulations

Stability of zika protein formulations was determined by incubating purified Zika protein prepared from BV1944 (EnvD with NTT introduced and His6 tag; SEQ ID NO:10) in formulations set forth in the table below at 4° C. and 25° C. for various timepoints (8 hours, 24 hours, and 4 weeks).

| | | Zika Antigen | Matrix M | Sample |
|---|---|---|---|---|
| DS Lot # | Antigen Formulation | (Conc. μg/mL) | (Conc. μg/mL) | ID |
| 161129 | 25 mM sodium phosphate, pH | 50 | 0 | 161129 |
| (200L | 7.5, 300 mM sodium chloride, | | | (50Z) |
| batch # 5) | 1 mM EDTA, 0.03% PS20 | 50 | 100 | 161129 |
| | | | | (50 + 100M) |
| | | 50 | 50 | 161129 |
| | | | | (50 + 50M) |
| 161025 | 25 mM sodium phosphate, pH | 50 | 0 | 161025 |
| (200L | 7.2, 150 mM sodium chloride | | | (50Z) |
| batch # 2) | | 50 | 100 | 161025 |
| | | | | (50 + 100M) |
| | | 50 | 50 | 161025 |
| | | | | (50 + 50M) |

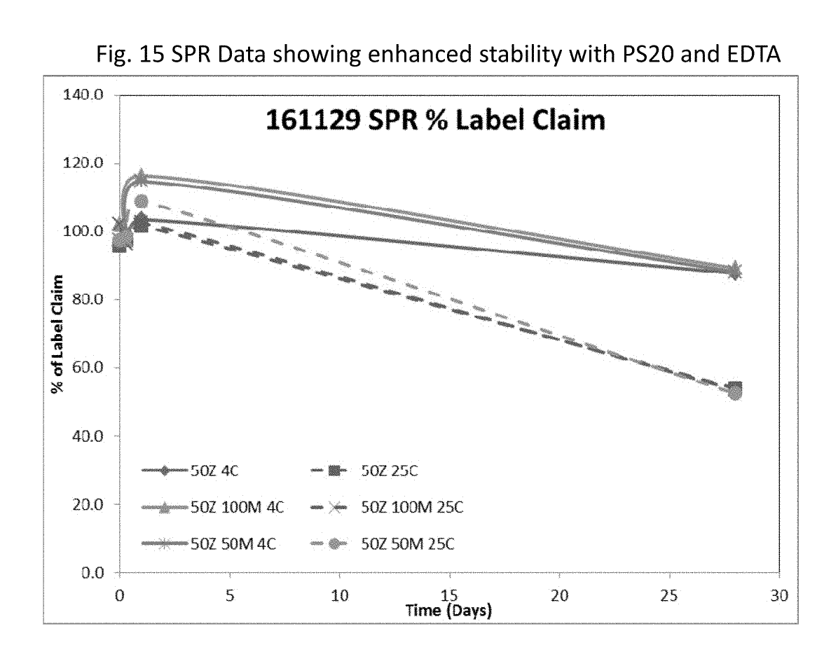

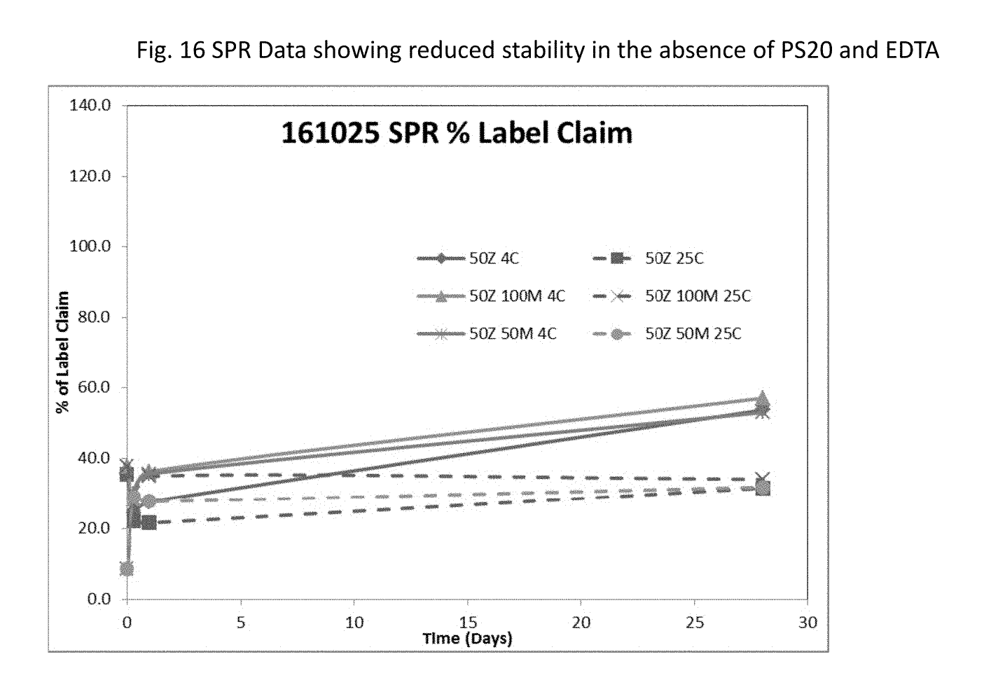

Protein stability was measured by A280 to determine intact Zika protein based on protein concentration. In addition, dimer stability was measured using Surface Plasmon Resonance (SPR) using an antibody to detect binding to the dimer. The results are shown below in the table below, and FIGS. 15 and 16, which show the SPR data profiles graphically. The data establish that PS20 and EDTA in the formulation is important to stability. While EDTA and PS20 preserved stability at a high percentage of the label claim amount (i.e., 50 μg/ml), protein amounts and dimer amount decreased dramatically in their absence. In fact, in the absence of PS20 and EDTA, a precipitate was observed (not shown) that likely accounts for most or all of the lost material compared to the label claim at time=0, with numbers under 40.0%, further underscoring the advantageous effect of PS20 and EDTA on protein and dimer stability.

FIG. 15 shows that, at 4 weeks, dimer stability is maintained at about 90% at 4° C. in the presence of PS20 and EDTA, and about 50% stability is maintained at 25° C. The data confirms that EDTA and PS20 in the formulation lead to enhanced stability. FIG. 16 shows that event at time 0 the Zika protein is unstable. Notably, the presence of Matrix M1 in the formulation did not reduce stability. These data support the use of a one-pot approach containing pre-mixed zika protein with Matrix M1 adjuvant.

| | | T = 0 | 8 hrs | 24 hrs | 4 W | | | T = 0 | 8 hrs | 24 hrs | 4 W |
|---|---|---|---|---|---|---|---|---|---|---|---|
| | A280 (μg/mL) | | | | | | A280 (μg/mL) | | | | |
| 161129 | 50 Z | 47.7 | 50.8 | 51.0 | 51.6 | 161025 | 50 Z | 38.0 | 29.4 | 30.7 | 29.7 |
| 4° C. | 50 Z + 100M | 49.8 | 51.1 | 52.6 | 49.6 | 4° C. | 50 Z + 100M | 31.0 | 33.2 | 33.0 | 32.0 |
| | 50 Z + 50M | 50.4 | 47.1 | 48.5 | 50.3 | | 50 Z + 50M | 36.6 | 28.4 | 30.0 | 29.5 |
| | SPR (μg/mL) | | | | | | SPR (μg/mL) | | | | |
| | 50 Z | 47.9 | 48.9 | 51.8 | 43.8 | | 50 Z | 17.7 | 12.2 | 13.8 | 26.8 |
| | 50 Z + 100M | 51.1 | 48.7 | 58.1 | 44.6 | | 50 Z + 100M | 18.8 | 14.1 | 18.1 | 28.5 |
| | 50 Z + 50M | 48.7 | 49.9 | 57.3 | 44.1 | | 50 Z + 50M | 4.2 | 15.1 | 17.8 | 26.5 |
| | A280 (μg/mL) | | | | | | A280 (μg/mL) | | | | |
| 161129 | 50 Z | 47.7 | 49.5 | 55.5 | 51.0 | 161025 | 50 Z | 38.0 | 33.0 | 26.4 | 31.1 |
| 25° C. | 50 Z + 100M | 49.8 | 52.0 | 49.8 | 52.1 | 25° C. | 50 Z + 100M | 31.0 | 33.2 | 33.0 | 38.9 |
| | 50 Z + 50M | 50.4 | 49.0 | 50.0 | 52.3 | | 50 Z + 50M | 36.6 | 29.4 | 34.4 | 31.3 |
| | SPR (μg/mL) | | | | | | SPR (μg/mL) | | | | |
| | | T = 0 | 8 hrs | 24 hrs | 4 W | | | T = 0 | 8 hrs | 24 hrs | 4 W |
| | 50 Z | 47.9 | 48.8 | 50.8 | 26.9 | | 50 Z | 17.7 | 11.0 | 10.8 | 15.8 |
| | 50 Z + 100M | 51.1 | 48.3 | 51.3 | 26.7 | | 50 Z + 100M | 18.8 | 13.4 | 17.5 | 17.0 |
| | 50 Z + 50M | 48.7 | 49.2 | 54.4 | 26.2 | | 50 Z + 50M | 4.2 | 14.6 | 13.9 | 15.9 |

### INCORPORATION BY REFERENCE

All patents, publications, journal articles, technical documents, and the like, referred to in this application, are hereby incorporated by reference in their entirety and for all purposes. Laroca et al., “Vaccine protection against Zika Virus from Brazil.” 536, 474-478 (2016) Dai et al., “Structure of the Zika Virus Envelope Protein and its Complex with a Flavivirus Broadly Protective Antibody.” Cell Host & Microbe, 19, 696-704 (2016).

1. A Zika virus polypeptide comprising
(a) an EnvD polypeptide, wherein the EnvD polypeptide comprises or consists of a polypeptide having at least 90% identity to SEQ ID NO:8; and, optionally,
(b) a heterologous amino acid portion C-terminal to the E80 polypeptide.

2. The Zika virus polypeptide of claim 1 wherein the heterologous amino acid portion comprises a protease cleavage site.

3. The Zika virus polypeptide of claim 2 wherein the protease cleavage site is selected from a group of sites cleaved by TEV protease, pepsin A, thermoylsin, furin, proteinase K, thrombin and trypsin.

4. The Zika virus polypeptide of claim 3 wherein the protease cleavage site is cleaved by TEV protease.

5. The Zika virus polypeptide of claim 1 wherein the heterologous amino acid portion comprises a tag.

6. The Zika virus polypeptide of claim 4 wherein the tag is selected from the group consisting of a FLAG-tag, a polyHis-tag, a Myc-tag, a Glutathione-S-transferase-tag, a Green fluorescent protein-tag, and a maltose binding protein-tag.

7. The Zika virus polypeptide of claim 6 wherein the tag is a polyhistidine tag and the tag is located C-terminal to the EnvD polypeptide.

8. The Zika virus polypeptide of claim 1 wherein the heterologous amino acid portion comprises the residual portion of a protease cleavage site and does not comprise a tag.

9. The Zika virus polypeptide of claim 1 wherein the EnvD polypeptide comprises an N-terminal PrM polypeptide having at least 90% identity to SEQ ID NO:9.

10. An immunogenic composition comprising the Zika virus polypeptide of claim 1, and a pharmaceutically acceptable carrier.

11. The immunogenic composition of claim 10 comprising dimeric polypeptides and wherein the dimer comprises dimer-specific epitopes.

12. The immunogenic composition of claim 10 wherein the composition comprises an adjuvant present in an amount effective to enhance the immune response to the Zika virus polypeptide.

13. The immunogenic composition of claim 12, wherein the adjuvant is selected from the group consisting of a mineral compound-based adjuvant, a bacterial adjuvant, an oil-based emulsion, an immunostimulatory complex (ISCOM), and a synthetic adjuvant.

14. The immunogenic composition of claim 12, wherein the adjuvant is Matrix-M1™ adjuvant.

15. A method of inducing an immune response comprising administering the composition of claim 10 to a subject.

16. The method of claim 15 wherein the subject is a human male or a female.

17. The method of claim 15 wherein the immune response comprises anti-Zika antibodies.

18. The method of claim 17 wherein the anti-Zika antibodies comprise a dimer-specific antibody.

19. The composition of claim 10 comprising
(a) a Zika polypeptide dimer, wherein the dimer contains two Zika virus polypeptides according to claim 1;
(b) about 20 mM to about 40 mM NaPO4, pH 7.2 to 7.5;
(c) about 200 mM to about 400 mM NaCl;
(d) about 0.02% to about 0.05% of a surfactant; and
(e) about 750 μM to about 1.5 mM EDTA.

20. The composition of claim 19 comprising
(a) a Zika polypeptide dimer, wherein the dimer contains two Zika virus polypeptides according to claim 1;
(b) about 25 mM NaPO4, pH 7.5;
(c) about 300 mM NaCl;
(d) about 0.03% PS20; and
(e) about 1 mM EDTA;

21. The composition of claim 10 wherein dimer stability, as determined by SPR using an anti-dimer antibody, is maintained at about 90% after 4 weeks storage at 4° C.


SEQUENCE LISTING (11 sequences)

SEQ ID NO: 1 - protein, 584 aa, Artificial Sequence
  ZIKV PrM.EnvD (67-69 DMA-NTT).TEV.His6
MVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDIVLEHGGCVTVMAQDKPTVDIELVTTTVSNMAEVRSYCYEASISNTTSDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGENLYFQGHHHHHH

SEQ ID NO: 2 - protein, 7 aa, Artificial Sequence
  TEV protease cleavage site
ENLYFQG

SEQ ID NO: 3 - protein, 577 aa, Artificial Sequence
  ZIKV PrM.EnvD.67-69NTT.His6
MVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDIVLEHGGCVTVMAQDKPTVDIELVTTTVSNMAEVRSYCYEASISNTTSDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGHHHHHH

SEQ ID NO: 4 - protein, 410 aa, Artificial Sequence
  Mature ZIKV PrM.EnvD (67-69 DMA-NTT).TEV.His6 post-TEV cleavage
IRCIGVSNRDFVEGMSGGTWVDIVLEHGGCVTVMAQDKPTVDIELVTTTVSNMAEVRSYCYEASISNTTSDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGENLYFQ

SEQ ID NO: 5 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSHFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDYSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATMGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLMAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 6 - protein, 4 aa, Artificial Sequence
  E antigen
DRGW

SEQ ID NO: 7 - protein, 404 aa, Artificial Sequence
  modified Zika virus Env protein with DMA
IRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSG

SEQ ID NO: 8 - protein, 404 aa, Artificial Sequence
  modified Zika virus Env protein with NTT
IRCIGVSNRDFVEGMSGGTWVDIVLEHGGCVTVMAQDKPTVDIELVTTTVSNMAEVRSYCYEASISNTTSDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSG

SEQ ID NO: 9 - protein, 167 aa, Artificial Sequence
  Zika virus PrM
MVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYS

SEQ ID NO: 10 - protein, 410 aa, Artificial Sequence
  Mature ZIKV PrM. EnvD 67-69 His6 with NTT
IRCIGVSNRDFVEGMSGGTWVDIVLEHGGCVTVMAQDKPTVDIELVTTTVSNMAEVRSYCYEASISNTTSDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGHHHHHH

SEQ ID NO: 11 - DNA, 1734 bp, Artificial Sequence
  ZIKV PrM.EnvD.67-69NTT.His6 DNA sequence
atggtgaccaggagaggctccgcctactatatgtacctggaccggaatgatgccggcgaggccatctccttccccaccacactgggcatgaacaagtgttacatccagatcatggacctgggccacatgtgcgatgccaccatgtcttatgagtgtccaatgctggacgagggcgtggagcccgacgatgtggattgctggtgtaataccacatctacatgggtggtgtacggcacctgccaccacaagaagggagaggcaaggcgcagccggagagcagtgacactgccttcccactctacccggaagctgcagacaagatctcagacctggctggagagcagggagtatacaaagcacctgatccgggtggagaactggatctttagaaatccaggattcgcactggcagcagcagcaatcgcctggctgctgggcagctccacctctcagaaagtgatctacctggtcatgatcctgctgatcgcccctgcctattccatcaggtgcatcggcgtgtctaatcgcgactttgtggagggaatgtccggaggcacctgggtggatatcgtgctggagcacggcggctgcgtgacagtgatggcccaggacaagccaaccgtggacatcgagctggtgaccacaaccgtgagcaacatggccgaggtgcggtcctactgctatgaggccagcatctccaacacgacctctgatagcagatgtcccacccagggcgaggcctacctggacaagcagagcgatacacagtacgtgtgcaagaggaccctggtggacaggggatggggaaatggatgtggcctgtttggcaagggctccctggtgacatgcgccaagttcgcctgttctaagaagatgaccggcaagagcatccagccagagaacctggagtacaggatcatgctgagcgtgcacggctcccagcactctggcatgatcgtgaacgacacaggccacgagacagatgagaatagggccaaggtggagatcacacctaacagcccacgcgccgaggccaccctgggaggatttggctccctgggcctggactgcgagcccagaacaggcctggacttctccgatctgtactatctgaccatgaacaataagcactggctggtgcacaaggagtggtttcacgacatcccactgccatggcacgcaggagcagatacaggaaccccacactggaacaataaggaggccctggtggagttcaaggatgcccacgccaagaggcagacagtggtggtgctgggcagccaggagggagcagtgcacaccgccctggcaggcgccctggaggccgagatggacggagcaaagggccgcctgtctagcggccacctgaagtgccggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggccggctgatcaccgcaaatccagtgatcacagagtctaccgagaacagcaagatgatgctggagctggaccctcccttcggcgatagctatatcgtgatcggcgtgggcgagaagaagatcacacaccactggcacagaagcggccatcaccaccaccatcactaa